(19) United States
(12) Patent Application Publication
(10) Pub. No.: US 2024/0017046 A1
(43) Pub. Date: Jan. 18, 2024

(54) DEVICES, SYSTEMS AND METHODS FOR TREATING THE SKIN

(71) Applicant: HydraFacial LLC, Long Beach, CA (US)

(72) Inventors: Dragan D. Nebrigic, Austin, TX (US); MohammedReza Emami, Corona, CA (US); Zahra Mardy Mamaghani, Austin, TX (US)

(21) Appl. No.: 18/035,723

(22) PCT Filed: Nov. 8, 2021
(86) PCT No.: PCT/US2021/072294

§ 371 Date: May 5, 2023

Related U.S. Application Data
(60) Provisional application No. 63/111,474, filed on Nov. 9, 2020.
(60) Provisional application No. 63/111,484, filed on Nov. 9, 2020.

(51) Int. Cl. A61M 35/00; A61M 1/00

(52) U.S. Cl. CPC A61M 35/003; A61M 1/774; A61M 2210/04

(57) ABSTRACT

According to some embodiments, a skin treatment system comprises a tower assembly including a manifold assembly, the manifold assembly comprising at least one fluid connector to secure at least one treatment fluid container, a handpiece assembly configured to hydraulically couple to the tower assembly, wherein the handpiece assembly is hydraulically coupled to the tower assembly using at least one fluid conduit, and a pressure source configured to deliver a separate fluid to the handpiece assembly, wherein the separate fluid is distinct from a fluid contained in the at least one treatment fluid container, wherein the handpiece assembly is configured to be hydraulically coupled to a vacuum source to remove spent fluid and other waste materials away from a distal end of the handpiece assembly during use, and wherein a pressure created by the separate fluid on skin tissue is configured to facilitate delivery of fluids delivered from the at least one treatment fluid container to the handpiece device deeper into a targeted skin surface of a subject.

### CROSS-REFERENCE TO RELATED APPLICATIONS

This application claims priority to U.S. Provisional Patent Application No. 63/111,474, filed Nov. 9, 2020, and U.S. Provisional Patent Application No. 63/111,484, filed Nov. 9, 2020. The contents of each of the aforementioned applications are incorporated by reference herein in their entireties.

### BACKGROUND

### Field

This application relates generally to skin treatment, and more specifically, to apparatuses, systems and methods for treating a person's skin.

### Description of the Related Art

Abrasion of the outer layer or epidermis of the skin is desirable to smooth or blend scars, blemishes, or other skin conditions that may be caused by, for example, acne, sun exposure, and aging. Standard techniques used to abrade the skin have generally been separated into two fields referred to as dermabrasion and microdermabrasion. Both techniques remove portions of the epidermis called the stratum corneum, which the body interprets as a mild injury. The body then replaces the lost skin cells, resulting in a new outer layer of skin. Additionally, despite the mild edema and erythema associated with the procedures, the skin looks and feels smoother because of the new outer layer of skin.

### SUMMARY

According to some embodiments, a skin treatment system comprises a tower assembly including a manifold assembly, the manifold assembly comprising at least one fluid connector to secure at least one treatment fluid container, a handpiece assembly configured to hydraulically couple to the tower assembly, wherein the handpiece assembly is hydraulically coupled to the tower assembly using at least one fluid conduit, and a pressure source configured to deliver a separate fluid to the handpiece assembly, wherein the separate fluid is distinct from a fluid contained in the at least one treatment fluid container, wherein the handpiece assembly is configured to be hydraulically coupled to a vacuum source to remove spent fluid and other waste materials away from a distal end of the handpiece assembly during use, and wherein a pressure created by the separate fluid on skin tissue is configured to facilitate delivery of fluids delivered from the at least one treatment fluid container to the handpiece device deeper into a targeted skin surface of a subject.

According to some embodiments, the treatment system comprises a second handpiece assembly, wherein the tower assembly is configured to provide the separate fluid to both the handpiece assembly and the second handpiece assembly. In some embodiments, waste conduits from both the handpiece assembly and the second handpiece assembly are in fluid communication with a single waste container.

According to some embodiments, the separate fluid is configured to be delivered continuously. In other embodiments, the separate fluid is configured to delivered intermittently (e.g., according to a pulsed pattern). In some arrangements, a pulsed pattern comprises a step-like pattern. In other embodiments, the pulsed pattern comprises an undulating (e.g., sinusoidal, other smooth, etc.), pattern.

According to some embodiments, the handpiece assembly comprises a piston or other pressure regulating device to regulate the flow of the separate fluid. In some embodiments, the pressure source comprises a pressure tank.

According to some embodiments, the at least one treatment fluid comprises at least two fluids, wherein the at least two fluids are not combined until they are within the handpiece assembly. In some embodiments, the handpiece assembly is configured to receive a vial, the vial configured to contain an additional treatment material.

According to some embodiments, the system is configured to collect data collected in association with use of the system and transmit said data to a network. In some embodiments, the network comprises a network over an internet connection.

According to some embodiments, the system is configured to operatively couple to an additional device to facilitate a treatment procedure. In some embodiments, the additional device comprises a device configured to detect at least one property of a skin surface being treated. In some embodiments, the at least one property of a skin surface comprises one or more of the following: skin texture, hydration or moisture level, elasticity, firmness, oiliness, redness or irritation, tone, luminosity and another skin property. According to some embodiments, the device comprises a camera or other imaging device.

According to some embodiments, the additional device comprises a device configured to provide treatment to a skin surface being treated. In some embodiments, the additional device comprises one or more of the following: a LED or other light-based handpiece or device, a device for providing thermal treatment, a lymphatic stimulation device and any other skin treatment device.

According to some embodiments, a method of treating skin comprises providing suction to a handpiece assembly of a skin treatment system, wherein providing suction creates a vacuum along a distal end of the handpiece assembly to engage at least a portion of a skin surface being treated, providing at least one treatment fluid to the distal end of the handpiece assembly from at least one fluid source, wherein providing suction to a handpiece helps remove at least a portion of the at least one treatment fluid delivered to the distal end of the handpiece assembly and other debris resulting from the skin treatment procedure, wherein providing suction further assists in providing at least one treatment fluid to the distal end of the handpiece assembly when the distal end of the handpiece assembly contacts a skin surface, and providing a separate fluid to the handpiece assembly using positive fluid pressure to move the separate fluid from a separate fluid source to the handpiece assembly.

According to some embodiments, the at least one fluid source comprises a bottle or other container positioned within a manifold. In some embodiments, the at least one fluid source comprises a vial that is secured to or along a portion of the handpiece assembly. In some embodiments, the at least one fluid source comprises both a bottle or other container positioned within a manifold and a vial that is secured to or along a portion of the handpiece assembly. In some embodiments, the separate fluid source comprises a pressure tank.

According to some embodiments, the separate fluid is configured to be delivered continuously. In some embodiments, the separate fluid is configured to delivered intermittently. In some embodiments, the separate fluid is configured to be delivered according to a pulsed pattern. In one embodiment, the pulsed pattern comprises a step-like pattern. In some embodiments, the pulsed pattern comprises an undulating or other smooth pattern (e.g., sinusoidal).

According to some embodiments, the handpiece assembly comprises a piston or other pressure regulating device to regulate the flow of the separate fluid. In some embodiments, the at least one treatment fluid comprises at least two treatment fluids, wherein the at least two treatment fluids are combined within the handpiece assembly.

According to some embodiments, the method further comprises collecting data related to a skin treatment procedure performed by the skin treatment system. In some embodiments, the data is related to at least one property of a skin surface being treated. In some embodiments, the at least one property of a skin surface comprises one or more of the following: skin texture, hydration or moisture level, elasticity, firmness, oiliness, redness or irritation, tone, luminosity and another skin property.

According to some embodiments, the method additionally comprises transmitting said data to a processor of the skin treatment system. In some embodiments, said transmitted data is configured to be provided to a network via a network connection of the skin treatment system. In some embodiments, the skin treatment system is configured to provide one or more suggested future treatment protocols for a subject based on an analysis of said data.

According to some embodiments, a skin treatment system comprises a console (e.g., tower) comprising a manifold assembly, wherein the manifold assembly includes a first receiving area and at least a second receiving area, wherein the first receiving area and the at least second receiving area are configured to receive a bottle containing a treatment material, a handpiece assembly configured to be in fluid communication with the manifold assembly using at least one fluid conduit, wherein the system is configured to selectively transfer a treatment material from a bottle secured to the first receiving area or the at least second receiving area to the handpiece assembly during a treatment procedure, at least one identification tag reader positioned at or near the first receiving area and the at least second receiving area of the manifold assembly, wherein the at least one identification tag is configured to communicate with an identification tag positioned on a bottle adapted to be secured to the first receiving area and the at least second receiving area, and at least one processor configured to receive information regarding the at least one identification tag from the at least one identification tag reader.

According to some embodiments, the at least one identification tag reader comprises a RFID tag reader. In some embodiments, the at least one identification tag comprises a RFID tag. In some arrangements, the at least one identification tag reader comprises a chip. In some embodiments, the at least one identification tag comprises an antenna.

According to some embodiments, the at least one identification tag is configured to both send data to and receive data from at least one identification tag reader. In some arrangements, the at least one identification tag is configured to modify the data contained therein based, at least in part, from data received by the at least one identification tag reader.

According to some embodiments, the at least one identification tag is located on a cap portion of the bottle. In some embodiments, the at least one identification tag comprises a flat or planar profile.

According to some embodiments, the at least one identification tag comprises a circular shape. In some embodiments, the at least one identification tag comprises an annular shape. In some embodiments, the at least one identification tag comprises a non-circular shape (e.g., square, rectangular, other polygonal, irregular, etc.).

According to some embodiments, the at least one processor comprises a first processor located at or near the manifold assembly. In some embodiments, the first processor comprises a motherboard or circuit board. In some embodiments, the at least one processor comprises a second processor that is operatively coupled to the first processor. In some embodiments, the second processor is operatively coupled to the first processor using at least one hardwired connection. In some embodiments, the second processor is operatively coupled to the first processor using at least one wireless connection.

According to some embodiments, the at least one processor is configured to be operatively coupled to a separate computing device. In some embodiments, the separate computing device is integrated into the treatment system. In one arrangement, the separate computing device comprises a touchscreen. In some embodiments, the separate computing device is not integrated into the treatment system. In some embodiments, the separate computing device comprises at least one of: a smartphone, a tablet, a laptop, a personal computer and another computing device or system.

According to some embodiments, the at least one processor is configured to be operatively coupled to a cloud computing infrastructure or platform. In some embodiments, the at least one processor is configured to provide an alert (e.g., warning, update, general information, etc.) to a user.

According to some embodiments, the user is an aesthetician performing a skin treatment procedure using the treatment system. In some embodiments, the user is a facility manager of a facility in which the treatment system is located. In some embodiments, the user is a supplier of the treatment system. In some embodiments, the user is a supplier of the bottles configured to be used in connection with the treatment system.

According to some embodiments, the at least one processor is configured to, based at least in part on data received from the at least identification tag reader, open or close a valve in fluid communication with a fluid line of the manifold assembly.

According to some embodiments, a bottle containing a treatment fluid and configured to be secured to a skin treatment system comprises a main body portion configured to receive a treatment fluid, a cap portion configured to be secured to the main body portion, and a nozzle extending from the cap portion, the nozzle configured to be positioned within and secured to a manifold assembly of a skin treatment system, wherein the cap portion comprises an identification tag, the identification tag being configured to provide data to a corresponding identification tag reader located on or along the manifold assembly of the skin treatment system, and wherein the cap portion cannot be removed from the main body portion without at least partially irreversibly damaging or destroying the connection between the cap portion and the main body portion.

According to some embodiments, the identification tag is flat or substantially flat. In some embodiments, the identification tag comprises a circular shape. In some embodiments, the identification tag comprises an annular shape. In some embodiments, the at least one identification tag comprises a non-circular shape (e.g., square, rectangular, other polygonal, irregular, etc.).

According to some embodiments, the identification tag comprises a RFID tag. In some embodiments, the identification tag comprises an antenna.

According to some embodiments, the identification tag is configured to both provide data to the corresponding identification tag reader and receive data from the corresponding identification tag reader.

According to some embodiments, the nozzle comprises at least one securement feature configured to help secure the bottle to the manifold assembly. In some embodiments, the at least one securement feature of the nozzle comprises an angled portion of the nozzle.

According to some embodiments, the main body portion and the cap portion comprise at least one plastic material. In some embodiments, the nozzle is integrated with a remaining portion of the cap portion.

According to some embodiments, the bottle further comprises a covering configured to be positioned on an open end of the nozzle. In some embodiments, at least one of the main body portion and the cap portion comprises a ratcheted design to help prevent separation of the main body portion and the cap portion without at least partially irreversibly damaging or destroying the connection between the cap portion and the main body portion. In some embodiments, both the main body portion and the cap portion comprise a ratcheted design to help prevent separation of the main body portion and the cap portion without at least partially irreversibly damaging or destroying the connection between the cap portion and the main body portion.

According to some embodiments, the identification tag is secured to the cap portion of the bottle using an adhesive.

### BRIEF DESCRIPTION OF THE DRAWINGS

These and other features, aspects and advantages of the present application are described with reference to drawings of certain embodiments, which are intended to illustrate, but not to limit, the present inventions. It is to be understood that these drawings are for the purpose of illustrating the various concepts disclosed herein and may not be to scale.

FIG. 1 illustrates a perspective view of a tower assembly and other components of a skin treatment system according to one embodiment;

FIG. 2 schematically illustrates a skin treatment system with various devices, components and features according to one embodiment;

FIG. 3A to 3D illustrate various embodiments of vacuum and/or positive pressure patterns in connection with a handpiece of a skin treatment system;

FIG. 4 schematically illustrates a hydraulic scheme for a skin treatment system according to one embodiment;

FIG. 5 schematically illustrates a hydraulic scheme for a skin treatment system according to one embodiment;

FIG. 6 schematically illustrates one embodiment of a hydraulic arrangement for a handpiece assembly for use in a skin treatment system;

FIG. 7 illustrates a perspective view of one embodiment of a handpiece assembly for use in a skin treatment system;

FIG. 8 illustrates a perspective view of one embodiment of a three-lumen fluid conduit for use in a skin treatment system;

FIG. 9 illustrates a coupling configured to secure a multi-lumen conduit to a corresponding recess or potion of a handpiece assembly according to one embodiment;

FIG. 10 schematically illustrates another embodiment of a hydraulic arrangement for a handpiece assembly for use in a skin treatment system;

FIG. 11 illustrates a monitor or other output device of a tower assembly according to one embodiment;

FIG. 12 illustrates the monitor other output device of FIG. 11 in a closed position;

FIGS. 13 and 14 illustrate different view of one embodiment of a tray or other storage device configured to be positioned along a top of a tower assembly;

FIG. 15 illustrates a perspective view of a handpiece assembly for a skin treatment system according to one embodiment;

FIGS. 16A and 16B illustrate different views of a tip having a rollerball according to one embodiment;

FIGS. 17A to 17E illustrate different views of the tip of FIGS. 16A and 16B;

FIG. 18 illustrates a perspective view of a skin treatment system according to one embodiment;

FIG. 19 illustrates various bottles containing treatment fluids and configured to be secured within a manifold assembly of the skin treatment system of FIG. 18;

FIG. 20 schematically illustrates a handpiece assembly being in fluid communication with a manifold assembly of a skin treatment system according to one embodiment;

FIG. 21 illustrates various views of one embodiment of a bottle configured to be positioned in a manifold assembly of a skin treatment system;

FIG. 22 illustrates a frontal perspective view of one embodiment of a bottle configured to be positioned in a manifold assembly of a skin treatment system, wherein the bottle comprises an automatic identification tag;

FIG. 23 illustrates an exploded view of the bottle of FIG. 22;

FIG. 24 illustrates a detailed view along the neck of the bottle of FIGS. 22 and 23;

FIGS. 25 and 26 illustrate different views of an automatic identification tag configured to be positioned on or along a bottle according to one embodiment;

FIGS. 27A to 27D illustrate different views of an upper portion of a bottle configured to be positioned in a manifold assembly of a skin treatment system according to one embodiment; and

FIG. 28 schematically illustrates a process diagram related to bottles with automatic identification tags for use in a skin treatment system according to one embodiment.

### DETAILED DESCRIPTION

Although the various embodiments of a handpiece assembly have specific relevance to a skin treatment system, the features, advantages and other characteristics disclosed herein may have direct or indirect applicability in other applications, such as, for example, medical devices, mechanical devices and/or the like.

Several embodiments of the inventions disclosed herein are particularly advantageous because they include one, several or all of the following benefits: (i) provide for enhanced delivery of treatment fluids to the skin of a subject; (ii) provide for deeper penetration of fluids into the skin of a subject; (iii) provide for delivery of fluids to the skin of a subject while reducing the likelihood of contamination; (iv) provide for the combination of two or more fluid streams at or near the tip-skin interface; (v) provide for enhanced collection of data regarding a skin treatment procedure; (vi) provide for enhanced treatment protocols based on data collection and processing; and (vii) provide for enhanced safety and other counterfeiting measures related to fluids delivered by skin treatment systems.

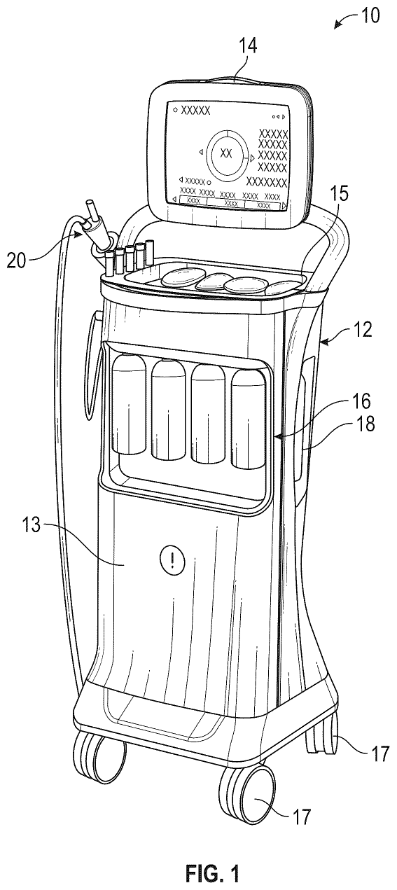

FIG. 1 illustrates one embodiment of a skin treatment system 10. As shown, the systems 10 can include a tower assembly 12 which includes various components and features of the system. For example, in some embodiments, the tower assembly 12 comprises a manifold assembly 16 for receiving one or more (e.g., 1, 2, 3, 4, more than 4, etc.) bottles or other containers of treatment fluids to be used in a skin treatment procedure. The tower assembly 12 can further include one or more waste canisters or other containers 18 that are configured to receive spent fluids, exfoliated or otherwise removed skin tissue and/or other waste products resulting from a skin treatment procedure. In some arrangements, one or more of the bottles, canisters or other containers that are designed and otherwise adapted to be secured to the tower assembly 12 can be replaceable, interchangeable and/or otherwise removable (e.g., for emptying, autoclaving or other types of cleaning, replacement, etc.).

The tower assembly 12 can further comprise one or more input and/or output devices (e.g., a touchscreen or other monitor 14), an outer housing or other exterior structure 13, tubing, one or more trays or other storage or mounting components 15, casters or other wheels 17, interior components (e.g., processor, memory, power source, tubing, valves and/or other hydraulic components, electrical wiring and other electrical components, etc.) and/or the like.

As discussed further herein, the tower assembly 12, and thus the skin treatment system 10, can be provided with one or more identification components, tools and/or other features that advantageously provide data or other information to the user, manufacturer or supplier and/or any other party or entity associated with skin treatment procedures performed by a particular system. For instance, the system 10 can be configured to identify one or more RFID tags or other identifiers (e.g., other electromagnetic identifiers, graphic or other visual identifiers such as QR codes, barcodes, alphanumeric codes, etc.) secured to or otherwise associated with a serum or other treatment fluid container. The tags or other identifiers can help ensure that the correct serums and other treatment serums are being utilized for a specific skin procedure or other protocol.

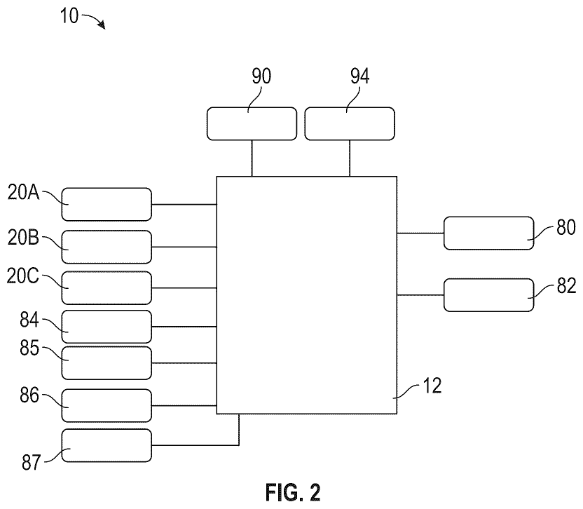

As illustrated schematically in FIG. 2, the treatment system 10 can include one more devices, components, systems, subsystems and/or other portions or features, as desired or required. In some embodiments, the system 10 comprises a tower assembly 12 that is configured to receive one or more other devices, components and/or features. For example, the tower assembly 12 can include one or more ports, couplings, access points, mounts and/or the like that can couple to and/or otherwise operatively connect (e.g., mechanically, electrically, via a data connection, etc.). Such coupling or other connections can include a physical connection and/or a wireless or non-physical connection.

In some arrangements, one or more components of the treatment system 10 can be configured to couple (e.g., physically, wirelessly, operatively, etc.) to the tower assembly 12. As illustrated in FIG. 2, the system 10 can include one more handpiece assemblies 20A, 20B, 20C, other types of handpieces (e.g., light emitting diode (LED) handpieces, masks or other devices) 90, 94, microscopes or other imaging devices (e.g., 80, 82), other skin treatment or assessment devices or tools (e.g., thermal treatment handpieces or devices, skin property measurement tools, etc.) 84, 85, Internet or other networking components or systems (e.g., IoMT components) 86, 87 and/or the like, as desired or required for a particular application or use.

### Enhanced Schemes for Fluid Delivery to the Skin Surface

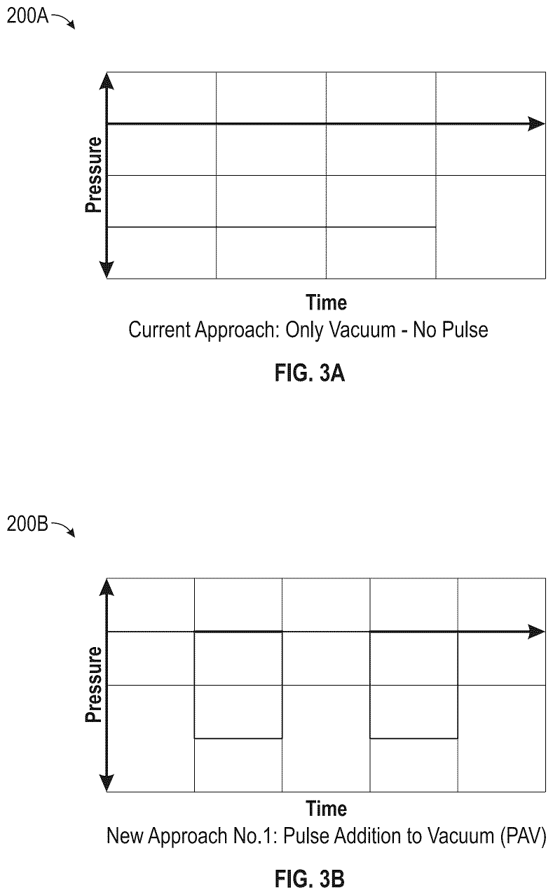

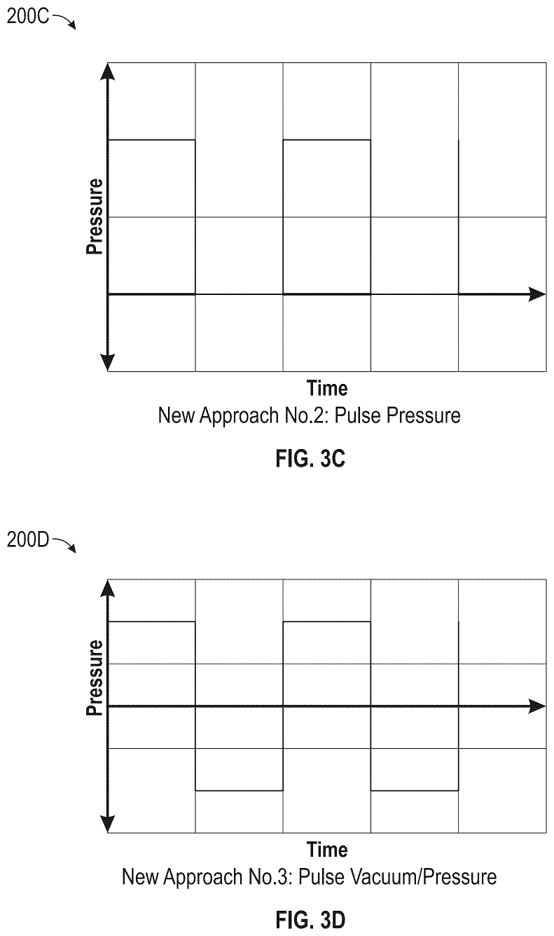

In some embodiments, a treatment system can be configured to provide one or more types pulsing effects and/or patterns to the tip or other distal portion that contacts targeted skin tissue of a subject undergoing a skin treatment procedure. FIG. 3A schematically illustrates a pressure pattern 200A along the tip or other distal end of a handpiece assembly. As shown, in such a configuration, a constant vacuum is applied by the handpiece along the tip or other distal portion (e.g., at or near the targeted skin surface being treated). Therefore, in such a configuration, no pulsing is utilized; however, the amount of vacuum or suction force that is generated and applied to the tip or distal end of the handpiece can be modified by a user in some embodiments. In some arrangements, for example, the tip or other distal end of the handpiece assembly can include a peripheral lip or other member that can be positioned along a subject's skin surface to form a seal (e.g., a complete seal, a partial seal, etc.). Once such a seal or other engagement is formed between the tip/distal end or portion and the subject's skin surface and the vacuum or suction force that acts upon the handpiece assembly is activated, the vacuum or suction force can draw toward the tip or other distal end or portion treatment fluid (e.g., fluid contained within a cartridge or other container that is secured to the handpiece assembly, a fluid manifold from a tower or console that is in fluid communication with the handpiece assembly, etc.).

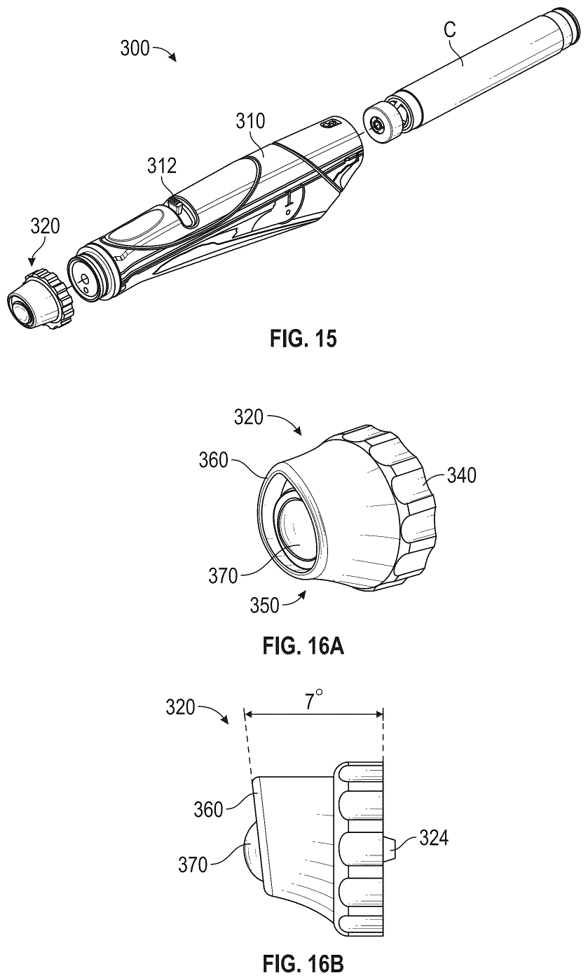

According to some embodiments, as illustrated in FIG. 15, a handpiece assembly 300 can include a flow adjustment member 312 along a main body portion 310 to permit a user (e.g., an aesthetician, someone else performing the skin treatment procedure, etc.) to easily adjust the flow of a treatment fluid that is being delivered to the subject. Such a mechanism can be separate and distinct from adjustments made to the vacuum or suction force applied to the tip or other distal end or portion of the assembly. Thus, in some embodiments, the amount of treatment fluid or other treatment material that is being delivered to the tip of a handpiece assembly can be adjusted by modifying a treatment fluid flow control feature (e.g., which can module a valve or other flow control mechanism related to treatment fluid flowrate), by adjusting the vacuum or pressure applied to the tip or other distal end/portion of the handpiece assembly or a combination of these strategies.

With reference to FIG. 3B, a skin treatment system can be configured to provide a pulsing effect 200B to a handpiece assembly that is only adapted to receive vacuum or suction force. Therefore, in some embodiments, the vacuum or suction source can be pulsed in order to modify the vacuum or suction effect delivered to the handpiece and tip, and thus, the targeted skin surface of the subject being treated. As shown in FIG. 3B, the pulsing pattern can include a step-like shape, wherein the vacuum or suction is activated to a particular level for a particular time period and then terminated. The vacuum or suction can then be re-activated after a desired “shut-down” time. The pattern can repeat according to a predetermined or set frequency and/or it can be modified (e.g., by the user, by an algorithm or strategy that is assisting with carrying out a particular treatment scheme, protocol or procedure, etc.), as desired or required.

FIG. 3C illustrates a different embodiment of a vacuum/pressure profile 200C being administered to a handpiece assembly. As shown, unlike the arrangement of FIG. 3B, positive pressure is being delivered to the tip or other distal portion/end of the handpiece assembly. Therefore, in some embodiments, the treatment fluid that is being delivered by the handpiece assembly to the targeted skin surface of the subject can be provided at a particular level of positive pressure.

With continued reference to the schematic of FIG. 3C, the treatment system can be configured to deliver treatment fluid to the tip or other distal portion of the handpiece assembly according to a positive pressure scheme. As schematically depicted in FIG. 3C, one or more treatment fluids can be delivered to the handpiece-skin interface using positive pressure. The pressure scheme can include a pulsed pattern, wherein the pressure is intermittently turned on and off according to a predetermined frequency or protocol.

FIG. 3D schematically illustrates yet another embodiment of a fluid delivery scheme that includes both negative pressure (e.g., suction or vacuum force) and positive pressure. As with other embodiments disclosed herein, the delivery of positive and/or negative pressure can be accomplished using a step-like function and/or an undulating (e.g., according to a sinusoidal or curved pattern or scheme), as desired or required.

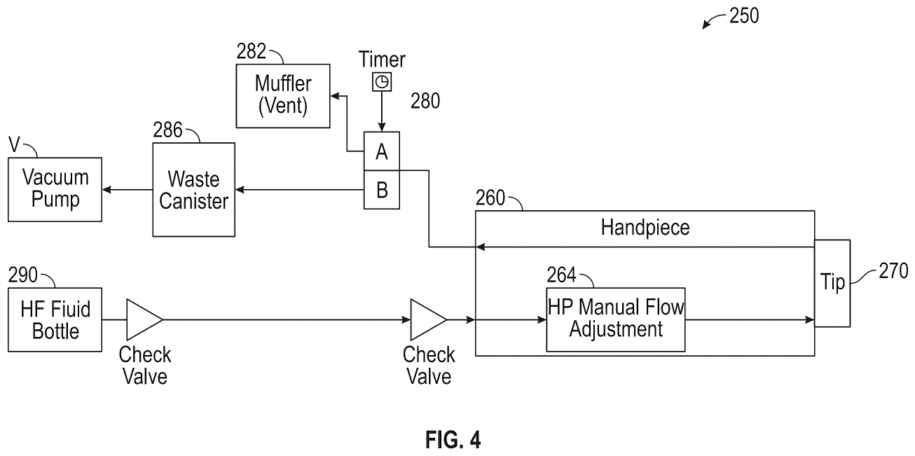

FIG. 4 schematically illustrates one embodiment of a handpiece assembly 260 for a skin treatment system 250 that is configured to selectively deliver negative pressure (e.g., suction or vacuum) and/or positive pressure to a targeted skin surface of a subject. As shown, the system 250 comprises a handpiece assembly 260 that includes a tip 270 located along the distal end of the handpiece assembly. The tip can be configured to selectively contact targeted skin tissue of a subject.

With continued reference to FIG. 4, the system 250 can include a valve mechanism 280 that allows the handpiece to selectively receive negative (e.g., suction or vacuum) and positive pressure. As shown, the valve or switch 280 can place the handpiece assembly 260 in fluid communication with a vacuum pump or other suction source V or can allow the handpiece assembly 260 to be hydraulically disconnected from suction or vacuum source V when the handpiece assembly is configured to deliver a treatment fluid to the tip under a positive pressure, as desired or required.

As shown, the system 250 can include one or more components to accomplish the desired or required scheme to the tip or distal end of the handpiece assembly. For example, in some embodiments, the system 250 can include a muffler or other vent 282, a waste canister (e.g., to capture any spent treatment materials, exfoliated or removed skin tissue and/or other waste materials, etc.) from the tip or distal end of the handpiece assembly and/or the like.

Further, as discussed herein, the handpiece assembly 260 can further include a flow adjustment feature, component, device and/or other mechanism 264 to provide the user with an opportunity to adjust the flow of one or more treatment materials form a treatment material source 290 to the tip or distal end 270 of the handpiece assembly. In some embodiments, the flow adjustment feature, component, device and/or other mechanism 264 comprises a valve or other flow control feature that regulates the flowrate of treatment fluid that is delivered to the tip or distal end. This can be separate from the regulation of vacuum/suction and/or positive delivery occurring within the handpiece.

Therefore, according to some embodiments, a handpiece assembly can be configured to deliver one or more treatment fluids and/or other materials from a fluid source (e.g., a cartridge positioned within the handpiece assembly, a manifold assembly, etc.) using vacuum/suction and/or positive pressure. In some embodiments, such vacuum/positive pressure delivery can occur in accordance with a step-line pattern (e.g., as schematically illustrated in FIGS. 3A to 3D), in accordance with a sloped or sinusoidal pattern and/or in accordance with any desired or required pattern.

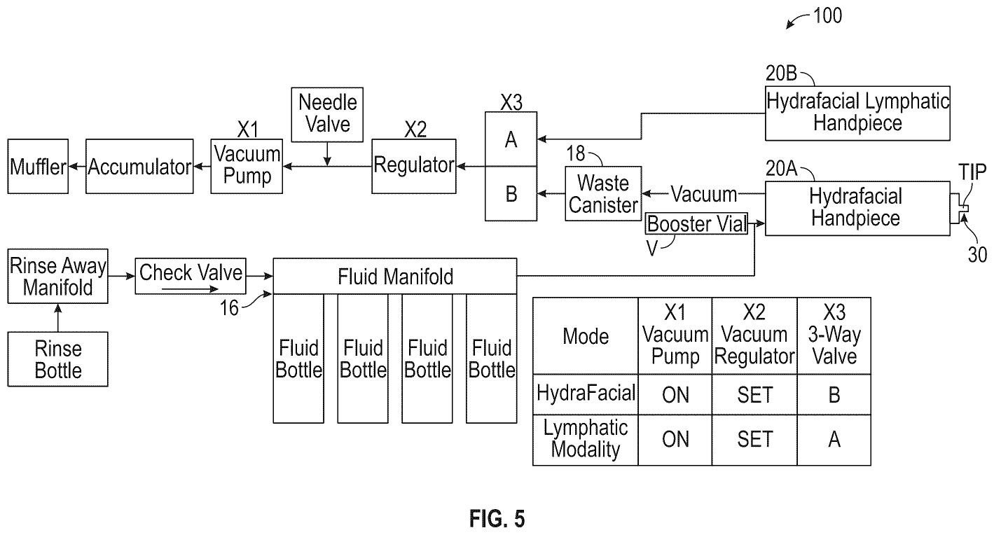

One embodiment of a hydraulic arrangement 100 for a skin treatment system is schematically illustrated in FIG. 5. As shown, the system can include two or more handpiece assemblies 20A, 20B that are configured to be used to perform one of a variety of treatment procedures. For example, in the illustrated arrangement, the system comprises a first handpiece assembly 20A that is configured to deliver one or more treatment fluids to the skin surface of the subject being treated. Spent fluids and other waste debris is removed from the tip of the handpiece assembly 20A and/or the skin surface of the subject using a suction system that is operatively coupled to one or more vacuum pumps. In some embodiments, the tip of the handpiece assembly 20A can be configured to cleanse the subject's skin tissue, exfoliate it, extract unwanted substances from it and/or hydrate it. Accordingly, in some embodiments, the tips secured to the end of the handpiece assembly 20A can include one or more of the following: abrading elements to help exfoliate skin tissue, fluid ports (to deliver serums and/or other treatment fluids to the skin), a peripheral lip (to selectively maintain suction with the targeted skin tissue) and/or any other components or features.

The depicted system additionally includes a second handpiece assembly 20B that may or may not be configured to deliver treatment fluids to the subject. By way of example, such a handpiece assembly 20B can include a lymphatic handpiece assembly that is configured to provide suction to a targeted skin surface. Such suction can be continuous or intermittent, and may or not may not be accompanying by fluid delivery through the handpiece assembly 20B. According to some embodiments, a procedure performed by a lymphatic handpiece assembly can provide one or more benefits to a subject, including, without limitation, removing toxic buildup from the subject's skin, assisting with the detoxification of the skin, decreasing inflammation, removing unwanted buildup and generally improving skin health.

With continued reference to the schematic of FIG. 5, the system can include a fluid manifold 16 with one or more fluid bottles or other containers. In the illustrated embodiment, the manifold 16 comprises a total of four bottles or containers. However, in other arrangements, the system can be configured to receive more than four (e.g., 5, 6, more than 6, etc.) or fewer than four (e.g., 1, 2, 3) bottles or containers, as desired or required. As discussed with reference to FIG. 1, such a manifold 16 can be positioned along an accessible surface or portion of a tower assembly 12 of a skin treatment system 10. Accordingly, for such configurations, a user can easily and quickly remove from and reinsert into a manifold assembly 16 bottles or other containers for use in a skin treatment procedure.

As illustrated schematically in FIG. 5, a conduit (e.g., tubing) can be used to place the manifold 16 in fluid communication with a handpiece assembly 20A. As discussed in greater detail herein, the handpiece assembly 20A can be further designed and otherwise configured to receive one or more vials V or other containers (e.g., directly, within a recess or other opening of the handpiece assembly). This can provide the user with additional flexibility, allowing the user to provide fluid from yet another fluid source (e.g., a vial V) through the handpiece assembly 20A to the distal tip 30 and the targeted skin tissue.

With continued reference to FIG. 5, the skin treatment system can include a vacuum or suction source (e.g., vacuum pump). The pump or other vacuum or suction source can be coupled to a vacuum or suction port and conduit of the handpiece assembly 20A to selectively create a suction force along the tip of the handpiece assembly 20A. Accordingly, such a suction force can help transfer spent serums and other treatment fluids that have been delivered to the skin, exfoliated skin tissue and other waste away from the skin surface being treated and to a waste canister 18. As noted herein, a waste canister 18 can be positioned in a tower assembly and/or any other portion of the skin treatment system. FIG. 5 illustrates additional devices and components that can be included in the system to facilitate with the transfer of fluids and other waste from the skin surface to one or more waste containers 18, including one or more of the following, regulators, valves (e.g., needle valves, mufflers and other noise reduction devices or components, accumulators and/or the like).

As noted above, the skin treatment system can be designed and otherwise adapted to accommodate one handpiece assembly or more than one handpiece assembly. For example, in accordance with the arrangement illustrated schematically in FIG. 5, the system can comprise a secondary handpiece assembly 20B. In the depicted arrangement, the second handpiece assembly 20B includes a handpiece assembly for lymphatic treatment in which vacuum or suction is delivered to the skin surface (e.g., intermittently, continuously, etc.). As shown, such a handpiece assembly 20B is in fluid communication with the vacuum pump or other suction pump. In some embodiments, a single vacuum pump or other suction source can be used to provide the desired or necessary suction force to two or more different handpiece assemblies 20A, 20B that are capable of being secured to a skin treatment system.

In order to facilitate the ability of the user to switch between different handpiece assemblies, a skin treatment system can include one or more valves or other flow regulating devices. For example, in FIG. 5, the system comprises a three-way valve X3 that advantageously places either the first handpiece assembly 20A or the second handpiece assembly 20B in fluid communication with the vacuum pump or other suction source. Such a valve or other flow regulating device X3 can be regulated manually or automatically (e.g., via a selection of a particular treatment protocol by the user using a touchscreen device or other input), as desired or required.

According to some embodiments, a skin treatment system can be configured to use vacuum or suction force to draw spent fluids and/or waste materials away from the skin surface being treated, while simultaneously causing treatment fluid (e.g., from a bottle or other container secured to a fluid manifold, from a vial or other container secured to or in fluid communication with the handpiece assembly, etc.) to be drawn to the skin surface of the subject. This can be accomplished by creating a closed suction system when the tip of the handpiece assembly is placed along a targeted skin surface being treated. For instance, the tip can include a peripheral lip that is configured to create at least a partial seal with the adjacent skin surface of the subject. Once such a partial seal is created and if a suction force is generated along the tip of the handpiece assembly (e.g., via a vacuum pump or other suction source that is in fluid communication with the handpiece assembly), a suction force created within an interior region defined by the tip and its peripheral lip or other boundary relative to the skin surface will help draw treatment fluid to the tip of the handpiece assembly.

However, in other embodiments, a treatment system is configured to deliver fluid (e.g., air, liquid) to the tip of the handpiece assembly in addition to the closed loop system described above. Such a delivery of fluid can assist with and provide benefits to a skin treatment procedure in one or more ways. For instance, fluid delivered using a positive fluid pressure to the skin surface (e.g., a mechanism above and beyond the use of only suction from a vacuum pump and a closed loop hydraulic design to move treatment fluid to the skin surface) via a handpiece assembly can assist in creating a desired fluid pattern along the tip-skin surface interface. Fluid delivered to the skin surface during a treatment procedure can include any combination of gas and liquid, as desired or required. For example, fluid delivered to the skin surface can include one or more of the serums or other treatment fluids that are used as part of a treatment protocol. In other embodiments, fluid that is delivered as a result of positive pressure to the skin surface can include air and/or other gases, water and/or any other liquid or gas, regardless of whether it is being used as part of a particular treatment procedure.

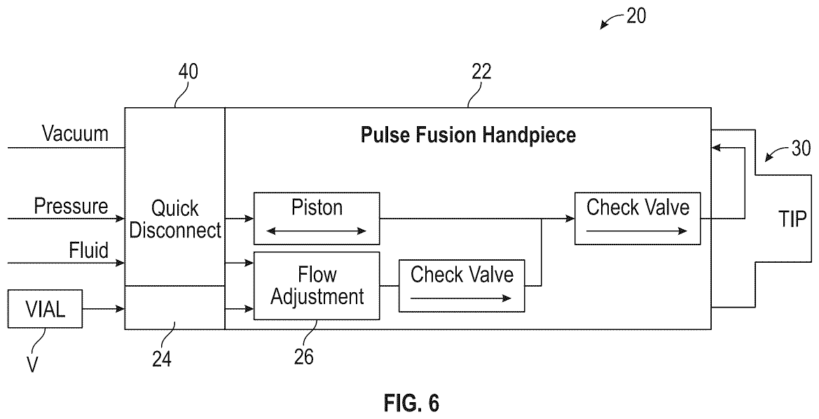

One embodiment of a handpiece assembly 20 that is designed and otherwise configured to deliver fluid using a positive pressure delivery scheme is schematically illustrated in FIG. 6. As shown, the handpiece assembly 20 can include a tip 30 that is configured to be selectively positioned by the user to contact a subject's skin surface (not shown). Further, in some embodiments, the handpiece assembly 20 comprises main body portion 22 that includes hydraulic components that facilitate the delivery of fluid under positive pressure to the tip 30.

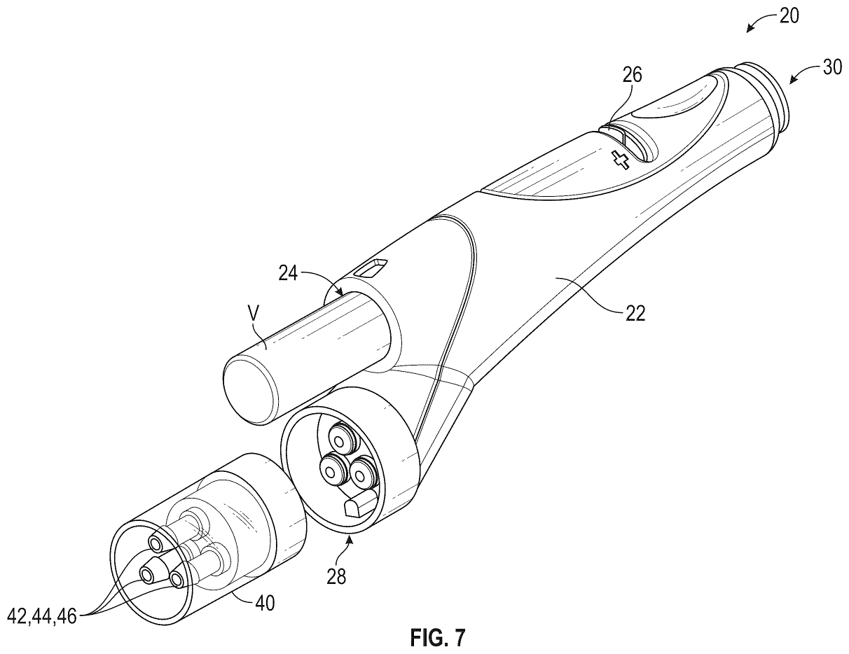

According to some embodiments, the handpiece assembly 20 comprises a first fluid pathway for fluid originating from a first fluid source (e.g., one or more bottles or other containers of a manifold) and a second fluid pathway originating from a second fluid source (e.g., a vial or other container that is secured to or otherwise placed in fluid communication with a portion 24 of the handpiece assembly). Such separate fluid streams can be collectively or individually controlled by a valve or other flow adjustment device (26). In some arrangements, such a valve or other flow adjustment device 26 is configured to be manipulated or otherwise controlled by the user while the user is performing a procedure. For example, as shown in the handpiece assembly illustrated in FIG. 7, the valve or other flow control device 26 can include or be coupled to a lever along the outside of the handpiece assembly that can be easily manipulated by the user.

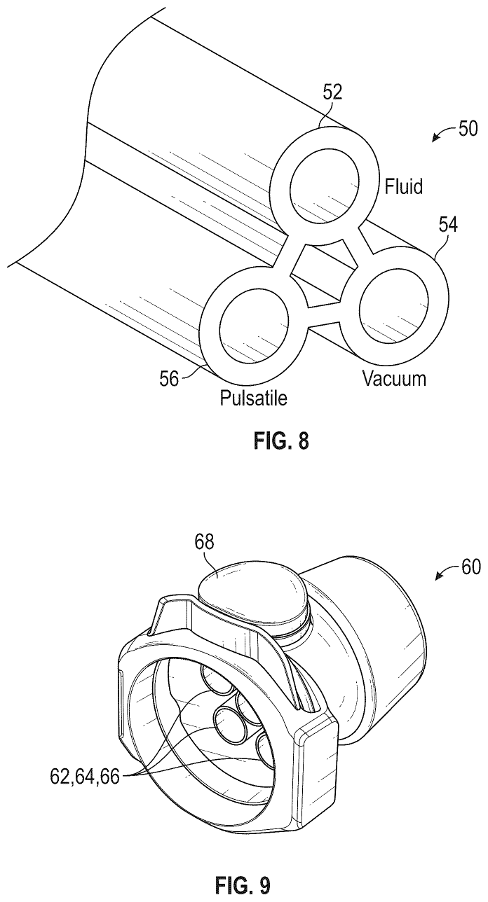

In some arrangements, the handpiece assembly 20 is further coupled to an additional fluid source that is configured to provide one or more fluids (e.g., a gas, a liquid, a combination thereof, etc.) using a positive delivery pressure to the tip 30. In some embodiments, as discussed in detail with reference to the embodiment of FIG. 8, such a separate fluid stream can be placed in fluid communication with a pressure tank or other pressure source of the system. Regardless of how the separate fluid stream is pressurized for delivery to the tip, such a separate fluid stream can be provided for any skin treatment system above and beyond other sources of treatment fluids (e.g., a manifold system, a vial secured to the handpiece assembly, etc.).

A separate fluid delivered to the tip of the handpiece assembly using a positive fluid source (e.g., a pump, a pressure tank, etc.) can provide one or more benefits to a skin treatment procedure. For example, the impact or pressure exerted by such fluid on the targeted skin tissue can help drive or push treatment fluids that are at the tip-skin interface deeper into the targeted tissue.

The separate fluid source that is delivered using a positive pressure source (e.g., pump, pressurized tank, etc.) to and through the handpiece assembly 20 can be delivered continuously or intermittently (e.g., using a pulsation scheme), as desired or required. In some embodiments, a pulsed delivery of such a supplemental fluid (e.g., air, other gas, liquid, etc.) can assist with pushing or driving treatment fluids deeper into skin tissue (e.g., vis-à-vis non-pulsated delivery of such fluid).

According to some embodiments, the separate fluid comprises air and is delivered to the handpiece assembly at a pressure of 0 to 200 psi (e.g., 0-200, 0-100, 100-200, 150-250, 0-50, 0-25, 25-50, 50-100, 100-150, 0-10, 0-20, 0-30, 0-40, 0-50, 10-50 psi, ranges and values between the foregoing, etc.). In some arrangements, the air other separate fluid is delivered to the handpiece assembly continuously. However, in other configurations, the air or other separate fluid is delivered to the handpiece in a pulsed manner. The rate of pulsation can be adjusted manually or automatically during the execution of a treatment procedure, as desired or required.

Further, as also illustrated in FIG. 6, various fluids being delivered through a handpiece assembly 20 can be combined at or near the tip 30, and thus, at or near the skin surface of the subject. As noted above, the fluids being combined can include liquids and/or gases originating from one or more bottles of a fluid manifold (e.g., of a tower assembly), a vial or other container secured to or otherwise placed in fluid communication with a handpiece assembly and/or a pressurized fluid source.

In some embodiments, combining two or more fluid streams at or near the tip or otherwise closer to the skin surface can provide some benefits and advantages. For example, it may be desirable or required to combine separate fluid streams at or closer to the skin surface of a subject for efficacy and/or safety reasons. In some embodiments, treatment fluids provide a better treatment result when they are combined along the tip-skin surface interface. Depending on the fluids being combined, it may be necessary to not combine the fluids too far in advance (e.g., form a time perspective, from a distance perspective, from a reactivity perspective, etc.) of delivery of the skin surface.

With continued reference to FIG. 6, the handpiece assembly 20 can include a coupling or other component 40 can is configured to easily and conveniently secure the various fluid conduits to the main body 22 of the assembly 20. In some embodiments, as discussed in greater detail below with respect to FIG. 7, the handpiece assembly 20 is configured to releasably couple to a quick disconnect assembly or component 40 along its proximal end. In one arrangement, such a quick disconnect assembly 40 is configured to couple the handpiece assembly 20 with various fluid streams, including, for example, a treatment fluid delivery line (e.g., from a manifold assembly), a positive pressure fluid line and a vacuum or suction line.

As illustrated schematically in FIG. 6, the handpiece assembly 20 can include a piston and/or one or more pressure regulating devices in fluid communication with the pressurized fluid line. Such a piston and/or other pressure regulating device can advantageously help regulate and/or maintain a desired pressure (e.g., or pulsating pressure effect) during use of the handpiece assembly. In some embodiments, the piston can help create the desired or required pulsation effect (e.g., between lower and higher pressure at a particular frequency). In other embodiments, the piston or other pressure regulating device can help maintain a constant or other desired pressure being delivered distally to the tip of the handpiece assembly.

FIG. 7 illustrates a perspective view of one embodiment of a handpiece assembly 20 comprising certain hydraulic features described above in reference to the schematic of FIG. 6 and/or equivalents thereof. The handpiece assembly 20 can include a distal end 30 that is configured to receive a tip (not shown). The depicted handpiece assembly 20 can include one or more of the improved design configurations discussed herein, including the ability to provide pressure at a particular level and/or in accordance with a desired or required pulsation effect.

With continued reference to FIG. 7, the handpiece assembly 20 can further comprise a recess or other opening 28 along its proximal end that is sized, shaped and otherwise configured to receive a coupling or other adapter 40 which is coupled to one or more fluid conduits. The adapter 40 can include a key or other alignment feature to ensure that the fluid conduits on either end of the adapter 40 are properly aligned. Further, the adapter 40 can include a tab and/or other quick release feature to enable the adapter to easily connect and disconnect to the corresponding recess 28 of the handpiece assembly 20.

FIG. 8 illustrates one embodiment of a three-lumen fluid conduit 50 that is configured to be secured to a handpiece assembly 20 and/or an intermediate adapter 40, as described above. As shown, the conduit 50 can include separate fluid passageways for fluid originating from a fluid manifold assembly 52, for suction or vacuum conduits 54 and a separate fluid stream (e.g., supplemental air, liquid or other fluid delivered to the handpiece according to a continuous or pulsating manner). In some arrangements, the various conduits of the conduit 50 are part of a monolithic or unitary design, as illustrated in FIG. 8. However, in other arrangements, one or more of the various fluid conduits 52, 54, 56 can be separate of other conduits, as desired or required. In some embodiments, a monolithic or unitary conduit assembly 50 is injection molded or otherwise manufactured as one member. However, the monolithic or unitary conduit assembly 50 can include separate conduits and/or other portions that are secured to each other after manufacture (e.g., using adhesives, clips or other securement devices or features, etc.)

FIG. 9 illustrates one embodiment of a coupling 60 that is configured to secure to a multi-lumen conduit, such as the one discussed herein with reference to FIG. 8. As shown, the coupling 60 can include one or more ports 62, 64, 66 to which the various conduits of a multi-lumen conduit 50 can connect and/or otherwise hydraulically couple. In some embodiments, the ports 62, 64, 66 of the coupling 60 can be sized, shaped and/or otherwise adapted to align and easily couple to corresponding conduits or other openings 52, 54, 56 of a multi-lumen conduit assembly 50.

As illustrated in the embodiment of FIG. 9, the coupling 60, which is configured to secure to the multi-lumen conduit 50 and releasably secure to a corresponding recess or other feature 28 of the proximal end of a handpiece assembly 20, can include a button or other releasing feature 68. Such a tab or other feature 68 can be configured to permit the coupling 60 to selectively secure to and release from the corresponding recess or other feature 28 of the handpiece assembly. In some embodiments, a tactile and/or audible confirmatory response is provided to the user when the coupling 60 has been properly secured to and/or release from the handpiece assembly 20 to help the user ensure that the handpiece assembly 20 is in proper fluid communication with the various fluid conduits of the corresponding multi-lumen conduit 50. Such a tab or feature 68 can also ensure that the coupling 60 is properly locked or otherwise secured to the handpiece assembly before a treatment procedure is commenced.

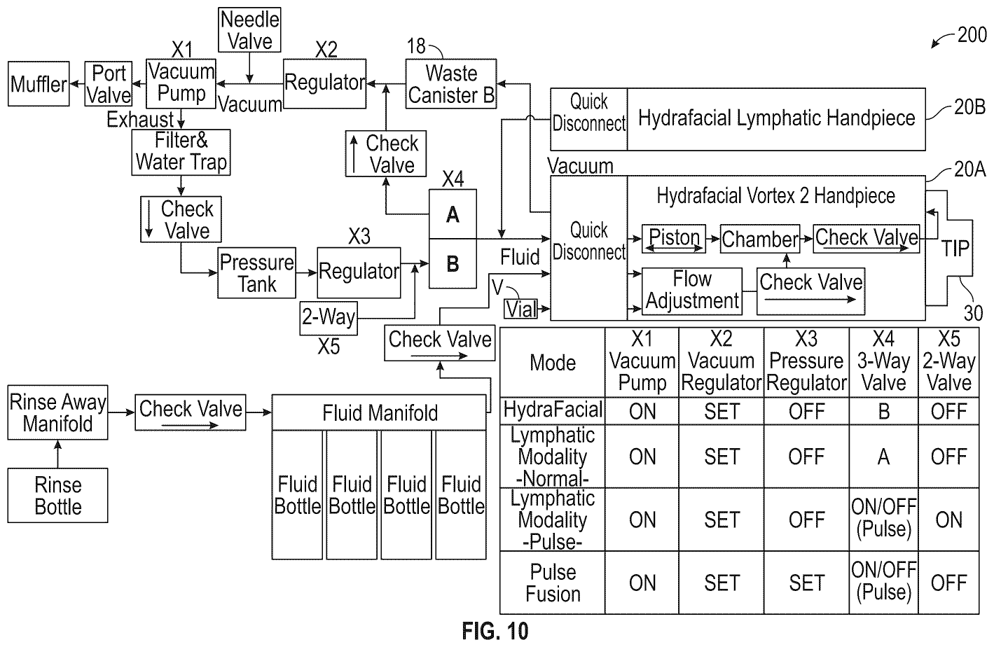

FIG. 10 schematically illustrates a hydraulic arrangement 200 for a skin treatment system that incorporates a handpiece assembly similar or identical to the one described above with reference to FIG. 6. In other words, the arrangement of FIG. 10 permits a user to deliver fluid (e.g., air, liquid, etc.) to the handpiece assembly 20A, 20B as a separate stream from any fluid being delivered to the handpiece assembly from the manifold assembly and any vials or other containers secured to or placed in fluid communication with the handpiece assembly.

As discussed above with reference to the embodiment of FIG. 5, the hydraulic arrangement is configured to permit a user of a skin treatment procedure to switch between various handpiece assemblies 20A, 20B (e.g., a fluid-based abrasion or exfoliation handpiece and a lymphatic handpiece assembly), while maintaining many common components and features. For example, the system illustrated in FIG. 10 (as with the system illustrated in FIG. 6) comprises a single waste canister 18 to which spent fluids, exfoliated skin tissue and other waste from either handpiece assembly is disposed.

With continued reference to FIG. 10, the system comprises a pressure tank or other pressurized vessel or system (e.g., pump) to help deliver fluid under positive pressure to one or more handpiece assemblies of the system. Thus, as discussed in greater detail above, pressurized air or liquid can be delivered to a handpiece assembly while one or more other fluids are being delivered to the tip of that handpiece assembly during a treatment procedure. Such additional pressurized fluid delivery can be delivered continuously or intermittently (e.g., in a pulsed manner), as desired or required.

A separate pressurized fluid line as disclosed herein can be advantageously hydraulically isolated from other fluid lines of the handpiece and/or of the overall system to reduce or eliminate the likelihood of cross-contamination of fluid streams (e.g., upstream of the skin surface being treated).

### Improved Tower Assembly

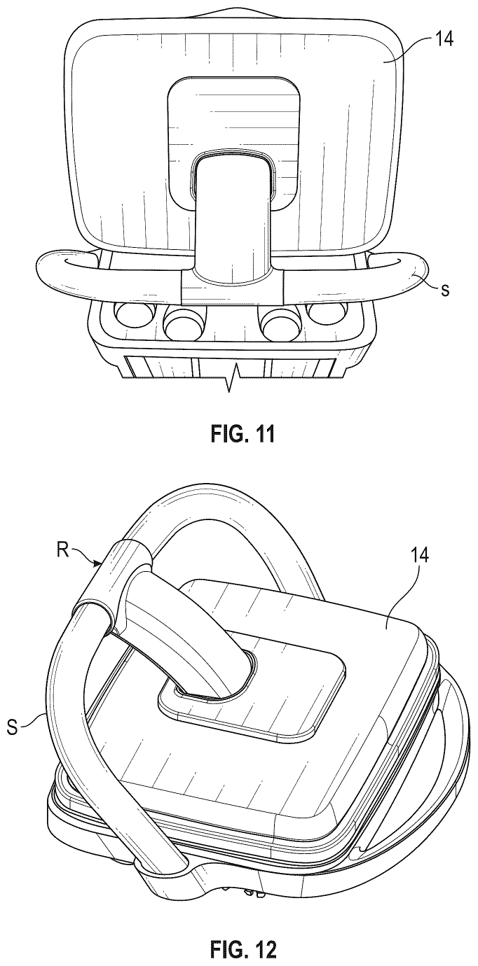

According to some embodiments, as illustrated in FIGS. 11 and 12, the monitor (e.g., touchscreen, other output, etc.) of 14 a tower assembly can be secured to one or more securement members S of the tower assembly. Securement members S can include rigid structural members, such as, for example, plastic and/or metallic tubing and/or the like. In some arrangements, the wiring (e.g., wire harness(es)) and other electrical connections for the monitor or other output 14 are routed, partially or completely, within an interior cavity of such securement members S. This can provide certain benefits to the tower assembly design, such as, for example, facilitating assembly and installation, simplifying the design, improving the optics and aesthetics of the unit and the like.

In some arrangements, the monitor or other display 14 is configured to be selectively rotated by a user from a closed position (as illustrated in FIG. 12) to an open position (as illustrated in FIG. 11). With reference to FIGS. 11 and 12, the monitor or other output 14 can be attached to one or more securement members S of the tower assembly at one or more hinge or other rotation points R to facilitate the movement of the monitor.

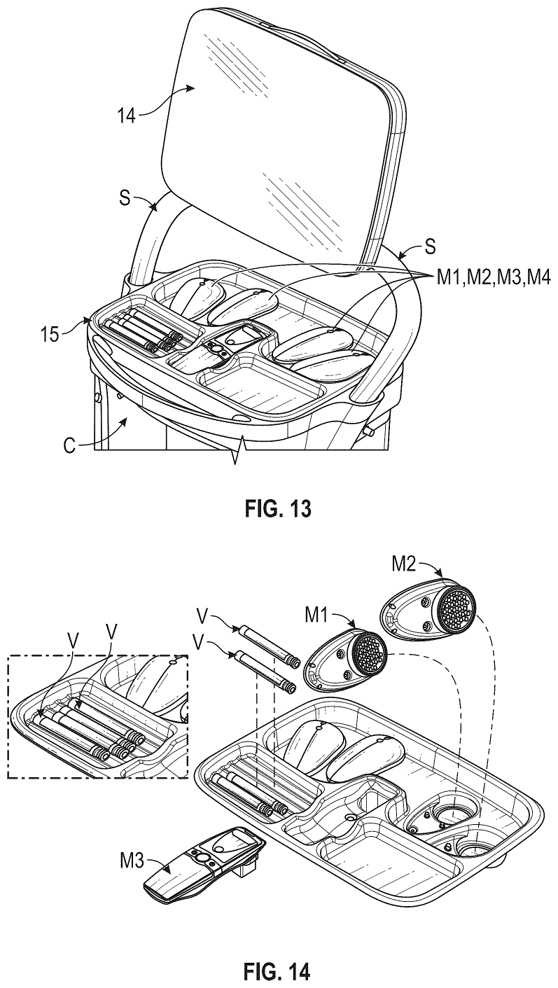

With reference to FIGS. 13 and 14, the tower assembly can include a tray or other storage device 15. As shown, such a tray 15 can be sized, shaped and otherwise configured to be placed along a corresponding recess or opening along the top of the tower assembly. In some embodiments, the tray 15 can include multiple compartments for receiving various components of the skin treatment system and other materials that may be used during the execution of a treatment procedure. For example, as shown in FIGS. 13 and 14, the tray 15 can include recesses or other opening for various modality units that may be provided with a skin treatment system, such as, e.g., LED handpieces M1, M2, a skin camera or other skin analyzing device M3 and/or the like. Further, the tray 15 can comprise appropriately sized and shaped slots to receive vials or other fluid containers V that may be secured to a handpiece assembly or otherwise used during a treatment procedure.

As shown in FIG. 12, in some configurations, the monitor or other display 14 of the tower assembly is configured to rotate downwardly (e.g., in a closed position) so as to cover the top of the tray 14. This can help ensure that any components or devices positioned on or in the tray can be safely and securely maintained in the tray when the unit is stored, transported or otherwise not in use.

### Handpiece and Tip Improvements

FIG. 15 illustrates a perspective view of handpiece assembly 300 configured to provide one or more treatment fluids to a subject's skin surface during a skin treatment procedure. As shown, the handpiece assembly 300 can comprise a main body portion 310 that is configured to removably receive within one or more recesses, chamber or other receiving areas a cartridge or other container C that contains a treatment fluid or material. A tip 320 can be configured to be positioned along a distal end of the main body portion 310. In some embodiments, the tip 320 can be configured to be removably positioned along the distal end of the main body portion. However, in other arrangements, the tip and the main body portion can be integrated (e.g., to form a unitary or monolithic structure), as desired or required.

With continued reference to FIG. 15, the handpiece assembly 300 can include one or more flow control features 312 that allow a user (e.g., an aesthetician) to regulate the amount of treatment fluid that is delivered to the tip from the cartridge or other container C that is placed in fluid communication with the handpiece assembly 300. In some embodiments, the flow control feature 312 can include a valve or other movable feature.

As illustrated in FIGS. 16A and 16B, the tip 320 can include a proximal portion 340 that is configured to secure to the distal end of the main body portion 310 of the handpiece assembly 300. In some embodiments, the proximal portion 340 includes a larger diameter or other cross-sectional diameter (e.g., to match exactly or substantially the corresponding diameter or other cross-section dimension of the distal end of the handpiece assembly). The tip 320 can include a rollerball or other moving structure 370. In some embodiments, such a rollerball or other moving structure 370 can be configured to facilitate the transfer of fluids from the cartridge or other fluid container C to the tip, and thus, the skin surface of the subject being treated.

With continued reference to FIGS. 16A and 16B, the tip 320 can include a peripheral lip 360 that surrounds and defines an interior region. The rollerball or other moving member or structure 370 can be positioned within the interior region. The tip can include one or more fluid inlets and/or outlets to permit treatment fluids to be delivered to the tip and/or spent materials, removed tissue and/or other waste materials to be removed from the tip and the corresponding handpiece/skin interface.

According to some embodiments, as illustrated in FIGS. 15, 16A and 16B, the distal end of the tip (e.g., the portion that is configured to contact a subject's skin tissue) comprises an angled or slanted distal end. In some embodiments, such an angled or slanted configuration can assist with the execution of a skin treatment procedure. As also illustrated in FIG. 16B, in some arrangements, the rollerball 370 can extend distally beyond the peripheral lip 360. However, in other embodiments, the rollerball 370 is aligned with or is proximal to the peripheral lip 360, as desired or required.

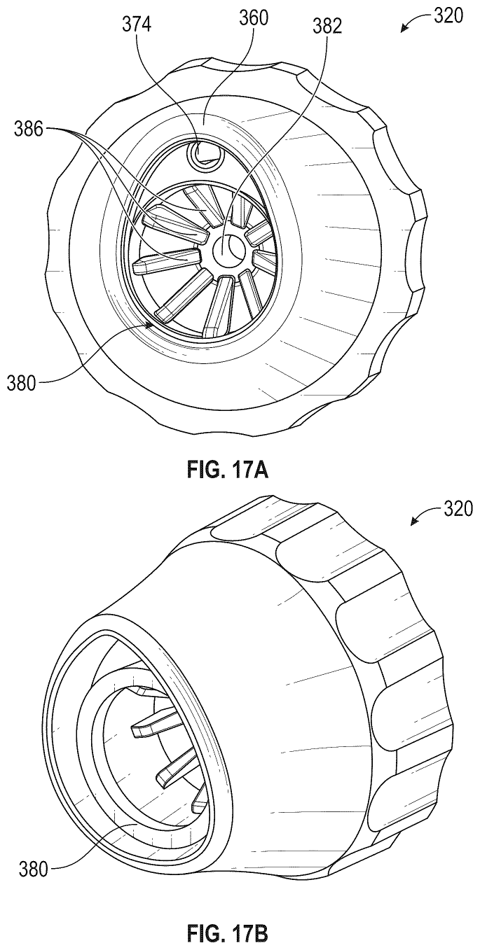

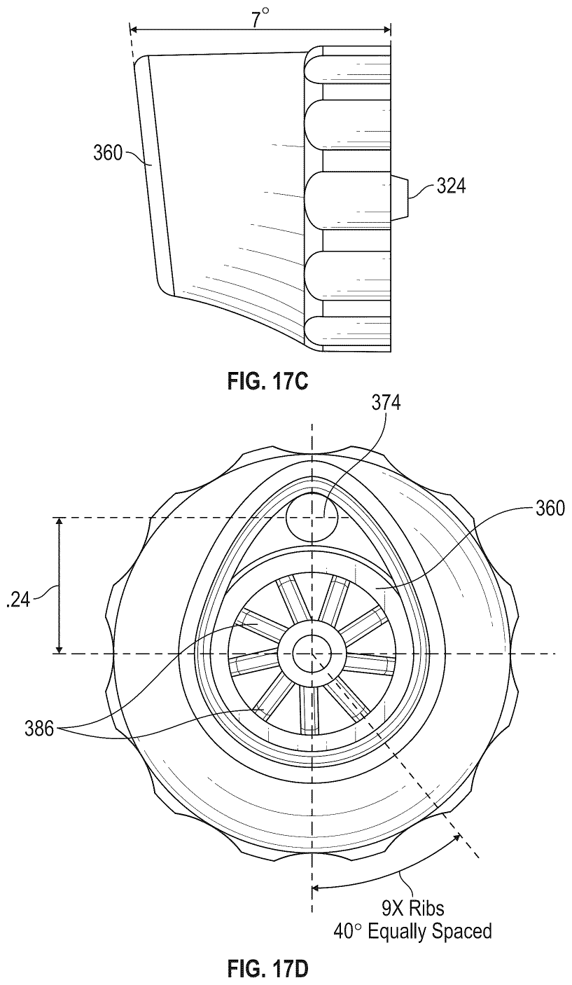

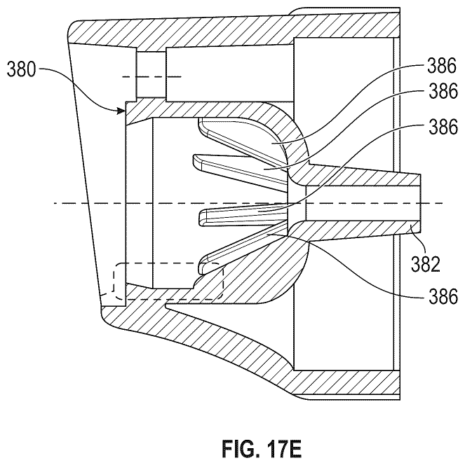

FIGS. 17A to 17B illustrate various views of the tip 320 shown in FIGS. 15, 16A and 16B. As shown, the tip 320 can comprise a partially spherical region or member 380 that is configured to receive and retain a rollerball. Such a region or member 380 can include one or more vanes, ribs or other members 382. In some embodiments, such ribs or other members can help retain a greater concentration of fluid (e.g., from the cartridge or other fluid source) adjacent or close to the rollerball. This can improve a skin treatment procedure, as fluid can be readily available adjacent the rollerball while the handpiece assembly is being manipulated and used by a user.

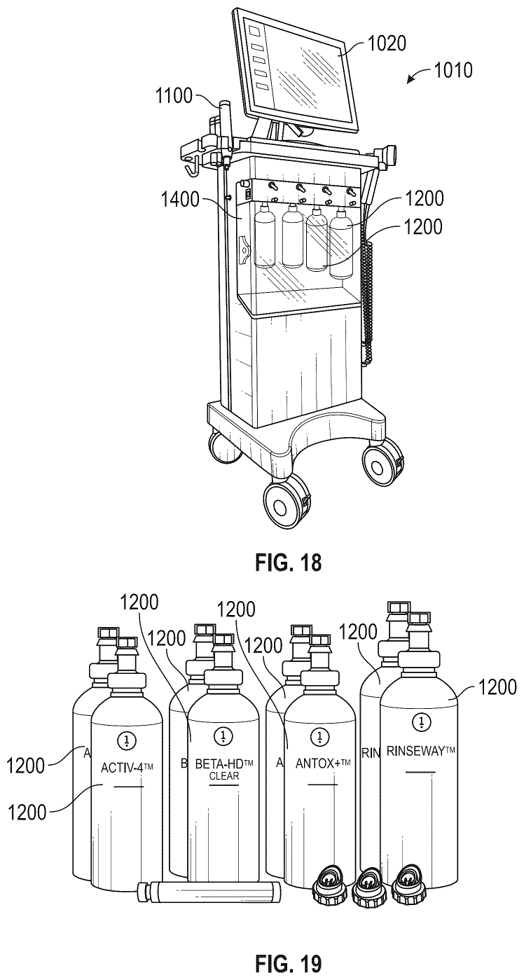

FIG. 18 illustrates a skin treatment system 10 according to one embodiment. As depicted in FIG. 18, the skin treatment system 1010 can comprise a handpiece assembly 1100 that is in fluid communication with one or more bottles or other fluid containers 1200. The handpiece assembly 1100 can be configured to deliver fluids (e.g., treatment liquids) to a distal end (e.g., the working end that contact skin) of the handpiece assembly during a treatment procedure. In some embodiments, the handpiece assembly 1100 comprises a tip along its distal end that is configured to be removed and replaced.

According to some embodiments, the handpiece assembly 1100 is in fluid communication with the various bottles or other containers 1200 positioned within and/or otherwise secured to the skin treatment system 1010 via one or more fluid conduits. As discussed in greater detail herein, the system 1010 can include a manifold assembly 1400 with which various bottles or other containers 1200 are placed in fluid communication when such bottles or other containers are properly secured to the system. The manifold assembly 1400 can be configured to direct liquid and/or other material contained in a particular bottle 1200 to the handpiece assembly 1100 (e.g., via one or more fluid conduits). In some embodiments, only liquid and/or other materials from a single bottle 1200 is directed to the handpiece assembly 1100 during a treatment procedure. However, in other arrangements, liquid and/or other materials from two or more bottles 1200 are simultaneously provided to the handpiece assembly. The delivery of liquids, other fluids and/or other materials can be regulated by a desired delivery pattern selected by a user (e.g., prior to and/or during a skin treatment procedure).

According to some embodiments, liquids, other fluids and/or other materials are transferred from one or more bottles or other containers 1200 secured to the system 1010 using the assistance of a vacuum or suction force. For instance, the system 1010 can include a vacuum or suction source (e.g., pump) and/or can be placed in fluid communication with a vacuum or suction source, as desired or required.

The handpiece assembly 1100 can include one or more fluid passageways (e.g., internal passageways) that are in fluid communication with the fluid conduit which connects the handpiece assembly 1100 with the bottles 1200 and/or the vacuum or suction source.

As noted above, a vacuum or suction force placed in fluid communication with a waste conduit can be configured to remove waste materials from the distal end of the handpiece assembly 1100 and help deliver liquids, other fluids and/or any other materials from a bottle or other container 1200 to the handpiece assembly-skin surface interface. When the distal end of the handpiece assembly is positioned against a subject's skin, suction created by the vacuum source can be transmitted to one or more fluid channels or conduits of the handpiece assembly 1100. Such a suction force created within the corresponding channels or conduits of the handpiece assembly can remain intact as long as the distal end of the handpiece assembly 1100 is maintained against or substantially against the subject's skin. Consequently, the suction force created by the vacuum source can be transferred to one or more fluid delivery channels of the assembly 1100, thereby transferring fluids and/or other materials from the bottle or other container toward the distal end of the handpiece assembly, and thus the skin tissue of the subject being treated.

In some embodiments, treatment liquids, other fluids and/or other materials can be delivered to the distal end of the handpiece assembly 1100 during use through one or more peripheral or other non-centrally located channels, conduits and/or other lines or fittings. For instance, such fluids and/or other materials can be routed through one or more internal channels of the assembly and/or waste conduits of the tip. In other arrangements, however, one or more of the fluid conduits or lines can be external to the handpiece assembly 1100, as desired or required.

In some embodiments, the tip or other distal end of the handpiece assembly includes an outer or peripheral lip or boundary that is configured to contact skin during use. The lip or boundary can form a seal when the handpiece is positioned along a skin surface (e.g., at certain angles relative to the skin surface). Accordingly, when the vacuum or suction source is activated, the seal can help draw liquids, other fluids and/or other materials from one or more bottles or other containers 1200 to the distal end of the handpiece assembly 1100, and thus, the skin surface of the subject being treated.

With continued reference to FIG. 18, the skin treatment system 1010 can include one or more additional components, devices and/or features, such as, for example, a cart (e.g., for housing and/or otherwise accommodating the various components of the system 1010), a touchscreen or other display, other input and/or output devices, other treatment devices and/or the like, as desired or required.

Additional details regarding the handpiece assembly and other aspects of the skin treatment system are provided in PCT Application PCT/US2014/024992, filed on Mar. 12, 2014 and published as WO 2014/151104 on Sep. 25, 2014. The entire contents of WO 2014/151104 are incorporated by reference herein and made part of the present application.

FIG. 19 illustrates an embodiment of various bottles 1200 shaped, sized and otherwise configured for placement in corresponding portions (e.g., bottle-receiving area) of a skin treatment system 1010 (see FIG. 18). The bottles can come in a variety of sizes, shapes and/or other configurations, as desired and/or required by a particular treatment system. Additional details regarding certain bottle designs are provided below.

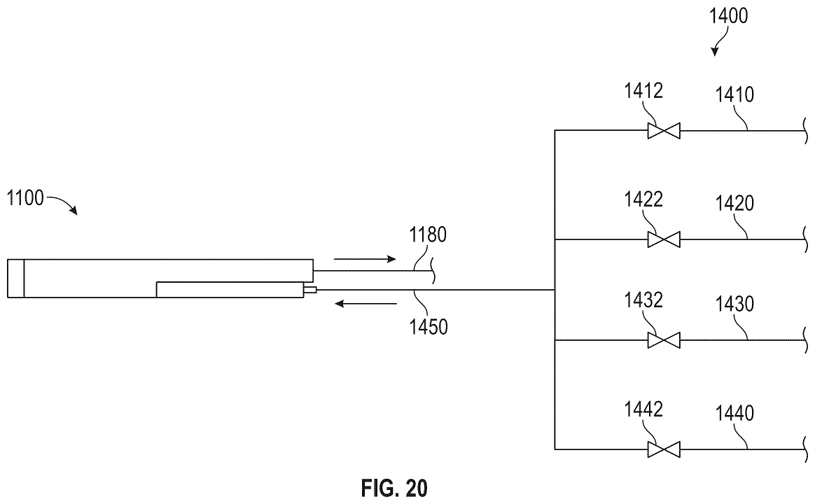

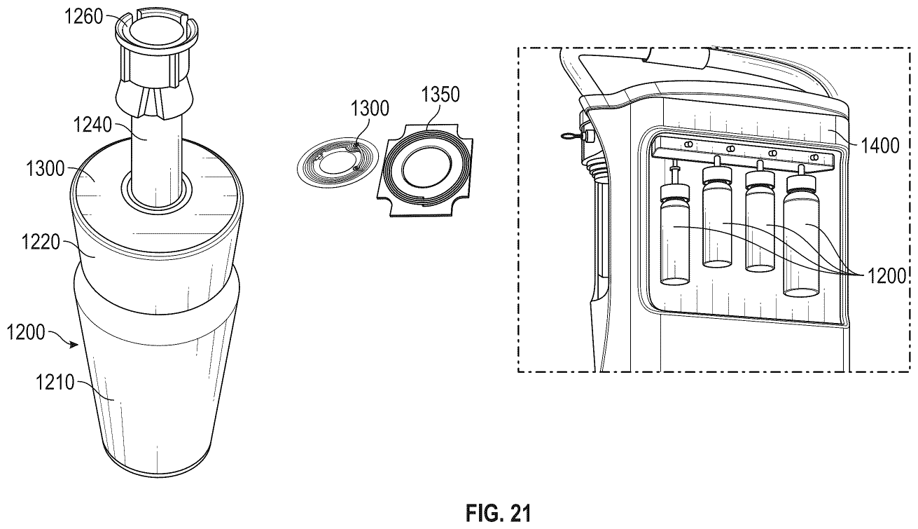

According to certain embodiments, as illustrated schematically in FIG. 20, a vial, cartridge or other container 1200 can be placed in fluid communication with a manifold system or assembly 1400 that comprises a plurality of individual conduits, fluid pathways or other fluid lines 1410, 1420, 1430, 1440. In turn, each of the individual fluid conduits or pathways 1410, 1420, 1430, 1440 can be in fluid communication with a bottle or other container (not shown in FIG. 20). For example, in some embodiments, such fluid conduits can be in fluid communication with corresponding containers of a tower system (see, e.g., FIGS. 18 and 21).

With continued reference to the schematic of FIG. 20, in some arrangements, the individual fluid lines or pathways 1410, 1420, 1430, 1440 are in fluid communication with a main fluid conduit 1450, which is configured to secure to a handpiece assembly 1100 (e.g., directly or indirectly). As shown in FIG. 20, one or more of the fluid conduits or pathways can comprise a valve 1412, 1422, 1432, 1442 or other flow control device or feature to selectively regulate the transfer of fluids and/or other materials to the handpiece assembly 1100. Such valves or other flow control devices can be configured to be manually and/or automatically controlled, as desired or required by the particular system. In the illustrated arrangement, the manifold system 1400 comprises a total of four fluid branches. However, a system can comprise more or fewer fluid branches (e.g., 1, 2, 3, 4, 5, 6, 7, 8, more than 8, etc.), as desired or required by a particular application or use.

According to certain embodiments, one or more of the fluid lines fluid conduits of the manifold system illustrated in FIG. 20 are configured to provide a serum, other treatment fluid and/or the like. Alternatively, however, one or more of the conduits can be configured to receive water (e.g., distilled, tap water, filtered, etc.), saline, other dilutants or dissolvents, other fluids and/or the like to the handpiece assembly 1100. Any of the fluids (e.g., water, saline, etc.) stored in a bottle 1200 and being delivered to the handpiece assembly (e.g., via a manifold assembly 1400) can be adapted to contact and dissolve, dilute, liquefy, soften and/or otherwise mix with one or more solids, gels and/or other materials positioned within or on various surfaces or portions of the handpiece assembly 1100 (e.g., tip). This can provide a convenient method of providing one or more materials at the skin-tip interface and/or any other location where such materials are desired or required.

In some embodiments, the vials, cartridges, bottles (e.g., used in towers or other manifold-systems) and/or other fluid sources can include any combination of skin tightening agents, platelet-rich plasma (PRP), exfoliation agents, peptides, bleaching agents, anti-acne agents, human growth factors, serums, salicylic acid, other anti-acne acids and materials, microcapsules, capsules, other time-release products and substances, human growth factors, cytokines, collagen, brightening or lightening agents, peptides, peeling agents, acids, antioxidants, matrix proteins, saline, water (e.g., distilled, tap water, filtered, etc.) and/or other liquids or substances, as desired or required by a particular application or use. In certain embodiments, a treatment protocol may require the use of one, two or more different cartridges for a specific procedure. Thus, vials or cartridges 1200 can be removed from or inserted into a handpiece assembly prior to or during a particular procedure. Alternatively, when a manifold system is being used to supply fluids to the handpiece assembly, one or more valves can be actuated (e.g., manually or automatically) to enable the desired fluid and/or other substance to be in fluid communication with the handpiece assembly.

FIG. 4 illustrates various views of bottles 200 configured to be positioned within a manifold assembly or other receiving portion 400 of a skin treatment system (e.g., a tower system). As shown, each of the bottles 200 can include a main body portion 210 and a cap portion 220. The cap portion 220 can be configured to include at least one surface or area that is sized, shaped and otherwise arranged to receive an automatic identification tag 300, such as, for example, a RFID tag or chip, a barcode, etc. Such tags can be used to advantageously store information regarding the specific bottle, vial or other container. For example, the tag can include information regarding the contents of the container, expiration date, manufacturing date, size, lot number, skin procedure with which the contents are intended to be used, other limitations or restrictions on use (e.g., counter-indications, adverse effects, other fluids with which the contents should not be combined, etc.).

The RFID or other identification tag can be read or otherwise detected (e.g., automatically, manually, etc.) by one or more readers or detectors of a manifold assembly 1400, a handheld assembly 1100 and/or any other portion of a tower system and/or a skin treatment system. For example, in some embodiments, such a reader can be placed at or near each station of a manifold system 1400 (e.g., adjacent the portion of the manifold to which the nozzle or top portion of the bottle or other container 1200 secures). Accordingly, the RFID or other type of reader can detect and identify the RFID tag of the bottle or other container. Likewise, a RFID or other type of reader can detect and identify the RFID or other identification tag (not shown) of a vial when such a vial or smaller container is properly positioned within the handheld assembly.

Therefore, in circumstances where the detected identifier is inconsistent with the proper, predicted, safe, appropriate and/or approved operation of the system, the system can be configured to prevent fluid from that vial or container from being used (e.g., by terminating the vacuum source, by maintaining a solenoid valve or other valve in the closed position, by otherwise preventing the flow of fluid from one or more of the bottles or other containers to the fluid delivery system of the manifold 1400, the handpiece and other portions of the tower and/or the skin treatment system, etc.).

The use of the RFID or other identification tags on the bottles, vials and/or other containers of the system can provide one or more other advantages or benefits. The collection of data regarding use of and/or related to the corresponding container (e.g., bottle, vial, etc.) can be gathered or otherwise collected to generate reports for billing, reordering and/or other purposes. In some embodiments, the number of times that a container can be removed and reinserted within a manifold or handheld assembly can be limited (e.g., 1, 2, 3, 4, etc.), as desired or required. For example, such limits can help prevent or reduce the likelihood of contamination of the fluid. In some embodiments, the automatic identification of the fluid container being secured to the system (e.g., manifold station, handheld assembly, etc.) can allow the system to determine if a rinse, flush and/or other steps or actions are required before the fluid from that container can be used and/or any other action can be taken in connection with use of the system.

According to some embodiments, the use of RFID or other identification tags can facilitate the execution of a particular skin treatment protocol by the system. For instance, the system can include various bottles containing fluids necessary to carry out any one of a number of various skin treatment procedures. For example, a treatment sequence can be configured for use in procedures for periodic or normal microdermabrasion treatment, anti-aging, anti-acne, skin lightening, oily skin treatment and/or the like. Each of the sequences, protocols or modes can include the delivery of one, two or more various serums and/or other fluids that are housed in the bottles or other containers 1200 in fluid communication with the manifold system 1400 and/or other portion of the skin treatment system.

In some embodiments, the skin treatment system comprises a touchscreen and/or other user interface (e.g., input and/or output device) 1020 (see, for example, FIG. 18) that allows a user to select from a number of several treatment protocols and/or other options. Such options can be pre-programmed (e.g., prior to the delivery of the system to a user). In some embodiments, the user is permitted to create a customized protocol, as desired or required. Regardless, once a specific treatment protocol or procedure is selected by a user, the system's control module can be configured to automatically recognize the bottles or other containers 1200 containing the serums and/or other liquids needed to complete a desired procedure have been loaded onto the system's manifold assembly 1400 (or any other portion of the skin treatment system). If the necessary products have been properly loaded (e.g., positioned with respect to the various receiving stations or portions of the system), the control module can initiate the corresponding skin treatment process or other protocol. Alternatively, if the control module determines that one or more fluids are missing (and/or that fluids included in a bottle already loaded onto the manifold system are improper, e.g., because they have expired, they have been recalled, or for any other purpose or reason), the system's control module can prevent the treatment protocol from being initiated. In other embodiments, the system's control module can take one or more other steps or action when such a recognition is made. For example, the system can provide a message to the user, informing the user of the error or other issue. In other embodiments, the occurrence of such an event provides the user with an opportunity to override any interruption and gives the user an opportunity to take certain actions to permit the process or protocol from resuming. For example, the user can be permitted to enter an override code (e.g., via a touchscreen or other input device of the system), to call a service center that can permit the process to continue and/or the like.

In some embodiments, RFID-based tags can be used to advantageously store information regarding the specific bottle, vial or other container. For example, the tag can include information regarding the contents of the container, expiration date, manufacturing date, size, lot number, skin procedure with which the contents are intended to be used, other limitations or restrictions on use (e.g., counter-indications, adverse effects, other fluids with which the contents should not be combined, etc.).

As illustrated in FIG. 21, according to some arrangements, the RFID or other identification tags can be read or otherwise detected (e.g., automatically, manually, etc.) by one or more readers or detectors 1350. Such detectors 1350 can be positioned on or along a manifold system 1400 and/or any other portion of a skin treatment system. For example, in some embodiments, such a reader can be placed at or near each station of a manifold assembly 1400 (e.g., above or adjacent the portion of the manifold to which the nozzle or upper portion of the bottle 1200 is configured to secure). Accordingly, the RFID or other type of reader can detect and identify the RFID tag (e.g., chip) or other identifier of the bottle or other container once such a bottle or other container is secured to the manifold. Likewise, a RFID or other type of reader can be configured to detect and identify the RFID chip or other identifier 1300 of a vial or other container when such a vial or other container (not shown) is properly positioned within the handheld assembly.

Accordingly, in circumstances where the detected identifier is inconsistent with the proper, safe, appropriate and/or approved operation of the system, the system can be configured to prevent fluid from that vial or container from being used (e.g., by terminating the vacuum source, by maintaining a solenoid valve or other valve in the closed position, etc.). As noted above, in some arrangements, such a prevention of fluid delivery can be temporary. For example, the stop can be temporary or reversible (e.g., by the user, by a supplier, etc.), as desired or required.

In some embodiments, the use of RFID or other identification tags 1300 on bottles, vials and/or other containers 1200 of the system provides one or more other advantages or benefits. The collection of data regarding use of the corresponding container (e.g., bottle, vial, etc.) can be collected to generate reports for billing, reordering and/or other purposes. In some embodiments, the number of times that a container can be removed and reinserted within a manifold or handheld assembly can be limited (e.g., 1, 2, 3, 4, etc.), as desired or required. For example, such limits can help prevent or reduce the likelihood of contamination of the fluid. In some embodiments, the automatic identification of the fluid container being secured to the system (e.g., manifold station, handheld assembly, etc.) can allow the system to determine if a rinse, flush and/or other preparatory steps are required before the fluid from that container can be used.

According to some embodiments, the use of RFID or other identification tags can facilitate the execution of a particular skin treatment protocol by the system. For instance, the system can include various bottles 1200 containing fluids necessary to carry out any one of a number of various skin treatment procedures. As illustrated and discussed herein, such bottles or other containers 1200 can be shaped, sized and otherwise designed and configured for placement in a manifold assembly 1400 of a skin treatment system (e.g., a tower of a skin treatment system).

In some embodiments, a treatment sequence can be configured as a core or basic fluid delivery sequence (e.g., for use in a periodic or normal wet microdermabrasion procedure). Other possible treatment modes or sequences include, but are not limited to, anti-aging, anti-acne, skin lightening, oily skin treatment, scalp treatment, treatment of other skin tissues (e.g., lips, eyes, etc.) and/or the like. Each of the sequences or modes can include the delivery of one, two or more various serums and/or other fluids that are housed in the bottles secured to the manifold system.

As noted herein, in some embodiments, the system comprises a touchscreen and/or other user interface 1020 (FIG. 18) that allows a user to select from a number of several treatment protocols and/or other options. Such options can be pre- programmed (e.g., prior to the delivery of the system to a user). In some embodiments, the user is permitted to create customized protocols, as desired or required. Regardless, once a specific treatment protocol is selected by a user, the system's control module can be configured to automatically recognize whether bottles or other containers 1200 containing the serums and/or other liquids necessary to complete the desired procedure have been loaded onto the system's manifold system.

If the necessary bottles or other containers 1200 have been properly loaded onto the various stations of the system, the control module can initiate the treatment process. Alternatively, if the control module determines that one or more fluids are missing (and/or that fluids included in a bottle already loaded onto the manifold system 1400 are improper, e.g., because they have expired, they have been recalled, or for any other purpose or reason), the control module can prevent the treatment protocol from being initiated.

In some embodiments, the system (e.g., via a touchscreen or some other interface, visual, audible, etc.) can be configured to alert the user that one or more of the required serums and/or other substances necessary or desired for a selected protocol (e.g., as included in a bottle or other container 1200 secured or otherwise positioned to a skin treatment system) are either missing or should not be used. Accordingly, the system can prompt the user to make the necessary changes in order to resume with the protocol.

In some embodiments, a treatment system is configured to permit a user to manually enter information about the contents of a bottle or other container loaded onto a station of the manifold system.

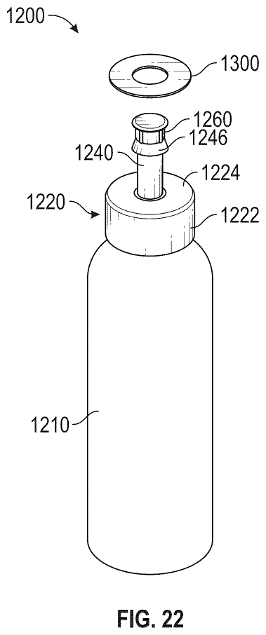

With continued reference to FIG. 21 and with further reference to FIG. 22, the bottle 1200 comprises a main body portion 1210 and a cap portion 1220. The cap portion 1220 can be configured to include at least one surface or area that is sized, shaped and otherwise arranged to receive an automatic identification tag 1300, such as, for example, a RFID chip, a barcode, etc. The bottle or other container 1200 configured to be secured to a manifold assembly 1400 and/or any other portion of a skin treatment system can further include an upper stem or portion 1240 (e.g., nozzle) that extends from and/or is part of the cap portion 1220.

As depicted in FIG. 22, the bottle 1200 can include a main body portion 1210 and a cap portion 1220. The cap portion 1220 can be configured to be secured to a top of the main body portion 1210. In some arrangements, as discussed in greater detail below, the cap portion 1220 is secured to the main body portion 1210 using an irreversible connection or attachment mechanism (e.g., such that the two portions cannot be reattached to one another without replacement of one or more components and/or portions). However, in other embodiments, the cap portion 1220 can be reversibly separated from and/or attached to the main body portion 1210 without the need to destroy, damage and/or replace one or more components or portions of the bottle assembly.

With further reference to FIG. 22, the cap portion 1220 of a bottle or other container 1200 can include a base 1222 and a nozzle 1240 extending from the base 1222. As shown, the base 1222 can include a flat or planar portion 1224 along its upper end or portion that is sized, shaped and/or otherwise configured to receive a RFID chip or other identification tag 1300. In some embodiments, the receiving area 1224 of the base 1222 has an identical or similar size and shape as the identification tag 1300. For example, in the illustrated arrangement, the receiving area 1224 and the identification tag 1300 include a circular or annular shape. However, in other embodiments, the shape of the receiving area 1224 and the identification tag 1300 can be different, such as, for example, oval, square, other rectangular, other polygonal, irregular, any other shape, etc., as desired or required.

The RFID or other identification tag 1300 can be secured to the receiving area 1224 of the base or any other area or portion of the cap portion or bottle using one or more adhesives. However, any other type of securement device, technology or method can be used, either in addition to or in lieu of adhesives, such as, for example, press-fit or friction fit connection, fasteners and/or the like.

In some embodiments, the bottles or other containers 1200 configured to be secured to a manifold assembly 1400 or other component or portion of a skin treatment system are provided to the user with the corresponding RFID chips or other identification tags 1300. In other words, the bottles or other containers 1200 can be supplied to the users by the supplier manufacturer or anyone else in supply chain with the tags 1300. However, in alternative embodiments, the tags can be provided by the user, as desired or required for a particular application or use.

With continued reference to FIG. 22, the cap portion 1220 can include a nozzle or other upper stem or portion 1240. As shown, such a nozzle 1240 can extend away from the receiving area 1224 of the base 1222. In some embodiments, the nozzle 1240 includes a cylindrical shape. A removable cap or other covering 1260 can be positioned along the distal end of the nozzle 1240 to help protect the nozzle against damage, contamination and/or the like. Further, the open end of the nozzle 1240 can include a removable seal, membrane or the like to provide additional protection. Further, the nozzle 1240 can comprise one or more engagement portion or feature 1246 along its length. In the illustrated embodiment, such an engagement feature 1246 includes a sloped portion extending away from the cylindrical shape of the nozzle 1240. However, in other arrangements, any other feature can be used. Regardless of its exact shape and configuration, the engagement portion or feature 1246 can be adapted to help secure (e.g., releasably secure) the nozzle 1240, and thus the bottle or other container 1200, to a manifold assembly 1400 and/or any other receiving area of a skin treatment system.

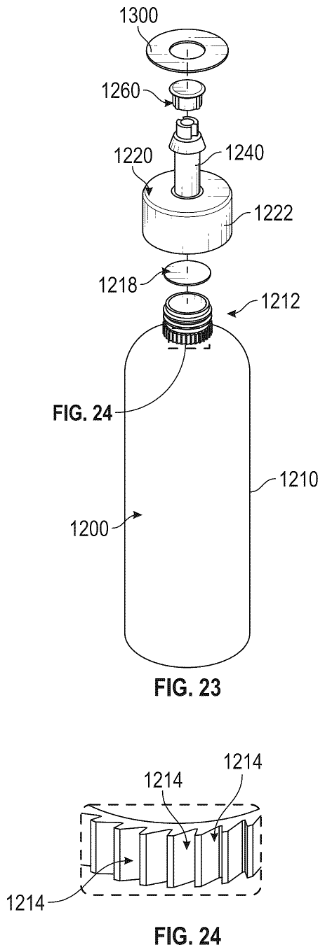

FIG. 23 illustrates an exploded view of one embodiment of a bottle 1200, such as the one illustrated in FIGS. 21 and 22, configured for use in a skin treatment system. As shown, the cap portion 1220 is configured to secure to the main body portion 1210 of the bottle using a threaded connection 1212. In some embodiments, a seal or member 1218 can be positioned between the cap portion 1220 and the main body portion 1210. Such a seal or member 1218 can be configured to be removed, pierced and/or otherwise compromised prior to and/or after the bottle 1200 has been secured to a manifold assembly or other portion of a skin treatment system.

As illustrated in FIG. 23 and the detailed view in FIG. 24, the bottle 1200 can include a ratcheted portion or area along its upper end. Such a portion or area can include a plurality of teeth or other sloped features 1214 that are sized, shaped and otherwise configured to engage corresponding teeth or sloped features along an adjacent portion along the interior of the cap portion 1220. As discussed in greater detail below, such a ratchet mechanism can help prevent removal of the cap portion 1220 from the main body portion 1210.

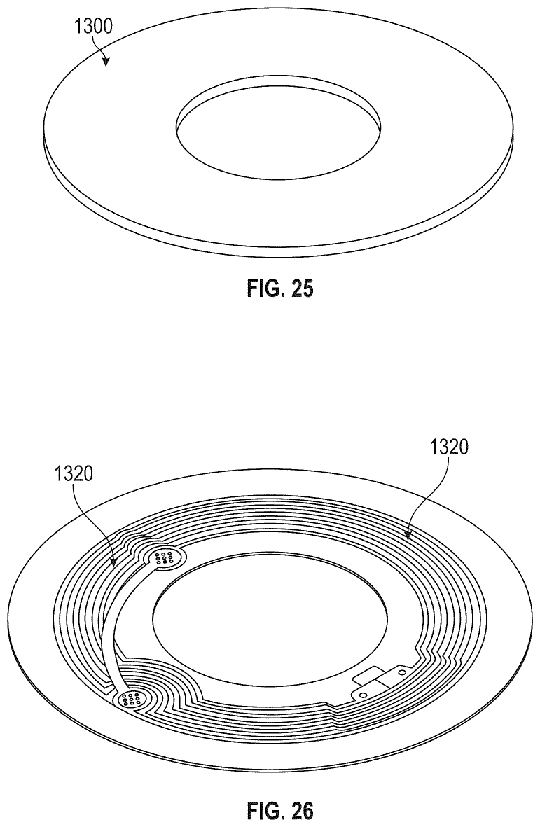

FIGS. 25 and 26 illustrate opposites sides of one embodiment of a RFID tag 1300. As shown, one side of the tag 1300 can include the necessary electronic components, including without limitation the chip (e.g., microchip), antenna and the like. The electronic components can be positioned on and/in (e.g., at least partially on, at least partially within, etc.) to the RFID tag 1300. In some embodiments, the opposite side of the RFID tag 1300 (e.g., the side illustrated in FIG. 25) can be configured to be secured to the bottle 1200 (e.g., the receiving area 1224 of the base 1222 of the cap portion 1220). In some embodiments, at least a portion of this opposite side can comprise an adhesive (e.g., a peelable adhesive strip or portion, glue or other adhesive, etc.). Other ways of securing the tag 1300 to the bottle can also be used, either in lieu of or in addition to adhesive (e.g., fasteners, press-fit or friction fit connections, etc.).

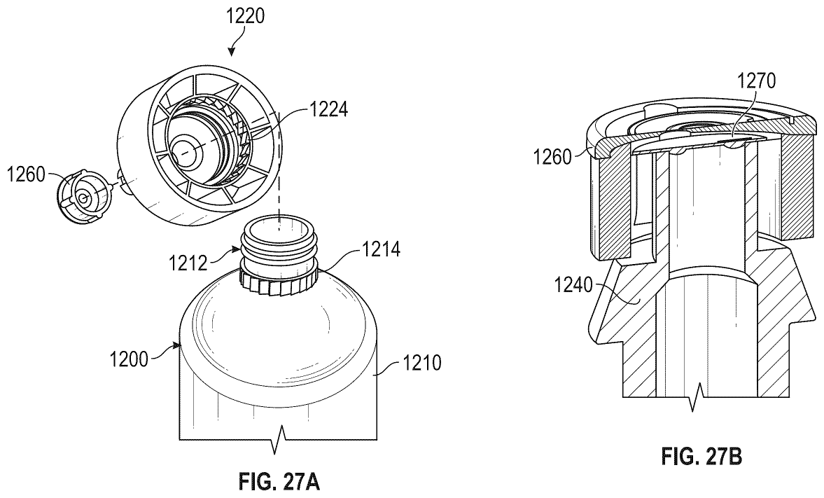

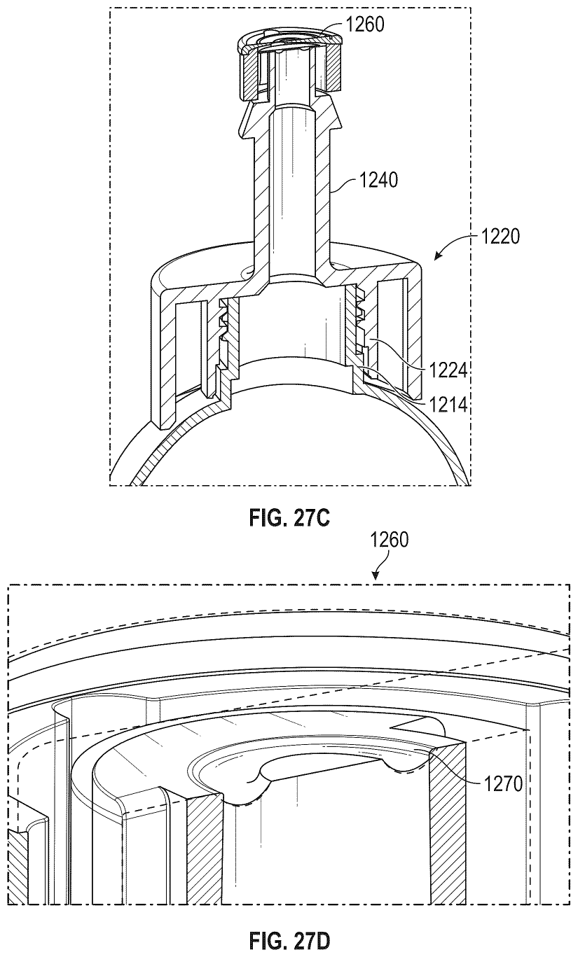

FIG. 27A to 27D illustrate various cross-sectional views of one embodiment of a bottle 1200 configured to be used with a skin treatment system. The bottle 1200 can be similar or identical to the bottle embodiments discussed herein with reference to FIGS. 21 to 24. As discussed herein and shown in FIGS. 27A to 27D, the cap portion 1220 can be configured to secure to the main body portion 1210 of a bottle 1200 using a threaded connection 1212.

With specific reference to FIG. 27C, in some embodiments, once the cap portion 1220 can be fully threaded onto the threaded portion 1212 of the main body portion 1210, the ratcheted or engagement portion 1214 of the main body portion can engage a corresponding ratcheted or engagement portion 1224 along the interior of the cap portion 1220. According to some arrangements, the ratcheted or engagement portions 1214, 1224 are located below (e.g., immediately below) the adjacent threaded portion of the main body portion 1210 and the cap portion 1220. In some embodiments, once the ratcheted or engagement portions 1214, 1224 of the main body portion 1210 and the cap portion 1220 are adjacent to one another, the cap portion 1220 cannot be rotated relative to the main body portion 1210 in a way that unscrews or otherwise separates the cap portion 1220 from the main body portion 1210. Accordingly, in such arrangements, the cap portion 1220 cannot be removed from the main body portion 1210 without at least partially damaging or destroying the cap portion 1220. As a result, the internal contents of the bottle 1200 cannot be accessed. This can prevent the intentional or inadvertent replacement, removal, refilling and/or other activities or actions associated with the contents of a RFID-tagged bottle 1200. Thus, the safety, effectiveness and predictability related to the use of the treatment system can be ensure and/or otherwise safeguarded.

As illustrated in FIGS. 27C and 27D, the cap portion 1220 can include a sealing member 1270 along its interior. The sealing member 1270 can include a relatively thin plastic member that is configured to span across the open end of the adjacent nozzle 1240. In some embodiments, the sealing member 1270 can be secured to one or more interior portions of the cap portion 1260 using any known technology or method (e.g., adhesives, ultrasonic welding, other welding or securement techniques, etc.).

In some embodiments, the sealing member 1270 is configured to provide isolation and other protection to the internal contents of the bottle or other container 1200. For example, the various bottles and/or other containers 1200 that are supplied to aestheticians or other users of a skin treatment system can include such a sealing member 1270 to prevent or reduce the likelihood of leaks, contamination and/or the like. In some configurations, once the cap portion 1220 has been removed from the nozzle 1240 of the bottle or other container 1200 (e.g., before securing the bottle or other container to a manifold assembly of a skin treatment system), the sealing member 1270 can be separated from the distal end of the nozzle 1240 to permit a user to secure the bottle or other container 1200 to a skin treatment system.

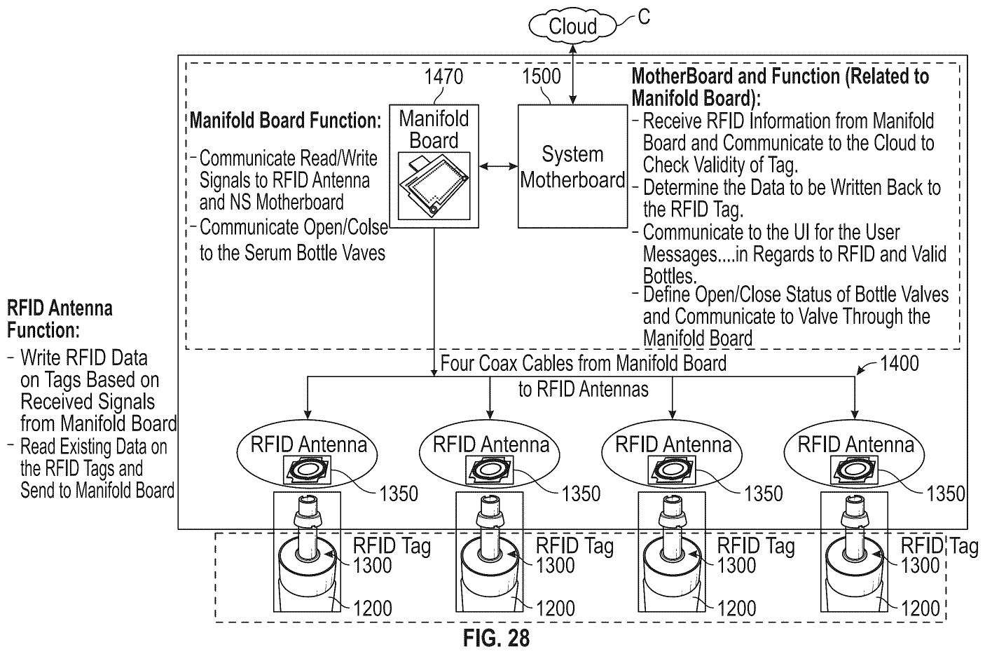

FIG. 28 schematically illustrates one embodiment of the manner in which RFID or other identification tags 300 secured to bottles or other containers 1200 interact with components and/or other portions of a skin treatment system. As shown, the various bottles or other container 1200 can be configured to be secured to a manifold assembly 1400 of a skin treatment system (e.g., a tower or console of a skin treatment system).

As illustrated in FIG. 28, any bottles or other containers 1200 secured to a manifold assembly 1400 and/or another portion of can be in data communication and/or can otherwise interface with a corresponding readers or detectors 1350. In some embodiments, such readers or detector 1350 comprise a RFID antenna or some other type of antenna or device that is configured to receive information from an identification tag. The readers or detectors 1350 can be strategically positioned near the location where the corresponding bottles 1200 are configured to secure to the manifold assembly 1400 or other securement of the bottle to the skin treatment is configured to take place.

With further attention to FIG. 28, the various RFID and/or other types of antennas or other data receiving devices 350 of the skin treatment system can be operatively coupled to one another (e.g., via one or more wired and/or wireless connections). For instance, in some arrangements, the RFID antennas or other data receiving devices 1350 are coupled to a manifold board, circuit or other hub 1470 using one or more coaxial cables. In some embodiments, each RFID antenna or other data receiving device 1350 is coupled to the manifold board, circuit or other hub 1470 using separate cables (e.g., coaxial cables). However, in other configurations, other types of cables and/or connections (e.g., wireless connections) can be used to operatively couple each of the antennas or other data receiving devices 1350 to the manifold hub 1470, as desired or required.

In some embodiments, the manifold board or hub 1470 can be configured to read signals received from the RFID tags 1300 of the bottles/other container 1200 secured to the manifold assembly 1400 and/or write signals to one or more other components, devices, etc. (e.g., a system motherboard, hub or processor, the RFID tag or other identification tag 1300 of the bottles 1200 and/or the like), as desired or required. For example, in some embodiments, the manifold board or hub 1470 is configured to write or otherwise provide data and/or other information from the various RFID or other identification tags 1300 of the bottles.

Such data and/or other information can include, without limitation, information regarding the specific serum, formulation and/or other treatment fluid or material included in a bottle, the date of manufacture, the expiration date of the contents of the bottle or other container, the volume of the treatment fluid or other material contained in the bottle or other container, the used and/or remaining volume of the treatment fluid or other material contained in the bottle or other container, the number of times or procedures a bottle or other container has been accessed, how many times a bottle can be removed and/or inserted into a manifold of a skin treatment system, the batch number and/or other unique identifier of the bottle or other container (e.g., for purposes of recalls and/or other safety controls) and/or the like.

Accordingly, the manifold board 1470 can keep track of data and/or other information regarding the bottles/containers 1200 and their contents. In some embodiments, the manifold board 1470 can be configured to transfer information to the RFID or other identification tags 1300 of the bottles 1200 (e.g., via the antennas 1350) to update certain data and/or other information for one or more of the bottles 1200. For example, the manifold board 1470 can provide a signal to a RFID tag 1300 of a bottle 1200 regarding how many times that bottle 1200 has been used (e.g., how many times and/or for how long the manifold has drawn fluid from the bottle). In some embodiments, the system is configured to estimate (e.g., empirically, accurately, roughly, etc.) how much of the volume of the treatment material in the bottle has been used and/or how much of the volume is remaining.

By updating data and/or other information on the RFID or other identification tag 1300 of the bottle 1200, such data/information can be retrieved and updated even if the corresponding bottle 1200 is removed from or reinserted in a manifold assembly. Thus, updated data and/or other information can be maintained about a bottle and its contents at all times. This can advantageously improve the safety, predictability, reliability and other factors associated with a skin treatment system and related procedure.

In some embodiments, the manifold board, hub or other processor 1470 is configured to open or close a valve or other flow control member associated with each bottle 1200. Thus, although not illustrated herein, a valve or similar flow control device or member can be positioned along each of the fluid lines of the manifold 1400. For example, in some embodiments, based at least in on information communicated to the manifold board or processor 1470, the system can be configured to disable use of a particular bottle or other container that has been secured to the manifold (e.g., initially, after a particular time duration or based on an event, etc.).

With continued reference to FIG. 28, the manifold board or processor 1470 can be operatively coupled to one or more other boards, processors, systems, devices and/or the like. For example, the manifold board 1470 can be in data communication with a system motherboard 1500. Such boards or other processors can be operatively coupled to one another using a wired connection (e.g., coaxial cable, another type of cable, etc.) and/or wirelessly, as desired or required.

According to some embodiments, the system motherboard or processor 1500 can be configured to receive data and/or other information from the manifold board or processor 1470, any other board or processor, a cloud and/or any other computing platform. Likewise, the system motherboard or processor 1500 can be configured to provide data and/or other information to the manifold board or processor 1470, any other board or processor, a cloud and/or any other computing platform.

In some arrangements, the system motherboard or processor 1500 is configured to determine what data should be written (e.g., updated) on the RFID tags or other identifiers 1300 of the bottles or other containers 1200. The system motherboard or processor 1500 can be configured to communicate with one or more user interface modules (e.g., a touchscreen of the tower/console assembly of the treatment system, a smartphone, a tablet or other computing device that is operatively coupled and/or in data communication with the system motherboard or processor 1500, etc.). Thus, the system motherboard or processor 1500 can provide messages to the user, can request information to be input and/or the like. In some embodiments, the system motherboard or processor 1500 can be configured to provide an alert to one or more users or entities associated with the operation of the treatment system. This can include, without limitation, data or information regarding the treatment material being used, confirmation that the bottle or other container being used is approved for the procedure, confirmation requests regarding the procedure and/or treatment materials and/or the like.

In some embodiments, the system motherboard or processor 1500 can provide instructions to the manifold board 1470 regarding the opening and/or closing of valves that are in fluid communication with the various bottles or other container secured to a manifold assembly. Such communications between boards, hubs and/or other processors, however, can occur in any manner desired or required by a particular system design. For example, the reading and/or writing of information, the transfer of instructions, user interface inputs and/or outputs and/or the like can be configured to occur using only a single board or processor. In other arrangements, two or more boards or processors can be used, as illustrated in the embodiment of FIG. 28.

In some embodiments, a treatment system can include one or more sensors or other detection devices to further enhance the data and/or other information associated with the use of the bottles or other containers secured to a manifold assembly 1400 of a treatment system. For example, the system can include a level sensor or a weight sensor to determine or estimate the amount of fluid that has been used or that is remaining in a particular bottle or container. Such sensors or other devices can be located at or near the manifold assembly 1400 and/or any other portion of the treatment system, as desired or required.

Various embodiments disclosed herein permit for the automatic detection and data/information transfer between the bottles and the treatment system (e.g., the manifold assembly). For example, RFID tags and corresponding readers are discussed herein. Further, one or more other ways of automatically or manually obtaining information regarding the bottles or other containers can also be used, either in lieu of or in addition to using RFID tags. For example, the bottles can include a barcode, QR code and/or other unique identifier that can read by a corresponding reader. Such a reader can be incorporated into the system (e.g., the manifold assembly, another portion of the console/tower, another portion of the system, a separate device that is operatively coupled to the system, etc.). In other embodiments, a keyboard or touchscreen can be used to manually enter information regarding the bottles or other containers being used.

Although several embodiments and examples are disclosed herein, the present application extends beyond the specifically disclosed embodiments to other alternative embodiments and/or uses of the inventions and modifications and equivalents thereof. It is also contemplated that various combinations or subcombinations of the specific features and aspects of the embodiments may be made and still fall within the scope of the inventions. Accordingly, it should be understood that various features and aspects of the disclosed embodiments can be combine with or substituted for one another in order to form varying modes of the disclosed inventions. Thus, it is intended that the scope of the present inventions herein disclosed should not be limited by the particular disclosed embodiments described above, but should be determined only by a fair reading of the claims that follow.

While the inventions are susceptible to various modifications, and alternative forms, specific examples thereof have been shown in the drawings and are herein described in detail. It should be understood, however, that the inventions are not to be limited to the particular forms or methods disclosed, but, to the contrary, the inventions are to cover all modifications, equivalents, and alternatives falling within the spirit and scope of the various embodiments described and the appended claims. Any methods disclosed herein need not be performed in the order recited. The methods summarized above and set forth in further detail below describe certain actions taken by a practitioner; however, it should be understood that they can also include the instruction of those actions by another party. The methods summarized above and set forth in further detail below describe certain actions taken by a user (e.g., a professional in some instances); however, it should be understood that they can also include the instruction of those actions by another party. Thus, actions such as “moving a handpiece” or “delivering a fluid” include “instructing moving a handpiece” and “instructing delivering a fluid.” The ranges disclosed herein also encompass any and all overlap, sub-ranges, and combinations thereof. Language such as “up to,” “at least,” “greater than,” “less than,” “between,” and the like includes the number recited. Numbers proceeded by a term such as “about” or “approximately” include the recited numbers. For example, “about 10 mm” includes “10 mm.” Terms or phrases preceded by a term such as “substantially” include the recited term or phrase. For example, “substantially parallel” includes “parallel.”

1. A skin treatment system comprising:
a tower assembly including a manifold assembly, the manifold assembly comprising at least one fluid connector to secure at least one treatment fluid container;
a handpiece assembly configured to hydraulically couple to the tower assembly, wherein the handpiece assembly is hydraulically coupled to the tower assembly using at least one fluid conduit; and
a pressure source configured to deliver a separate fluid to the handpiece assembly, wherein the separate fluid is distinct from a fluid contained in the at least one treatment fluid container;
wherein the handpiece assembly is configured to be hydraulically coupled to a vacuum source to remove spent fluid and other waste materials away from a distal end of the handpiece assembly during use; and
wherein a pressure created by the separate fluid on skin tissue is configured to facilitate delivery of fluids delivered from the at least one treatment fluid container to the handpiece device deeper into a targeted skin surface of a subject.

2-81. (canceled)
